(19) United States
(12) Patent Application Publication
(10) Pub. No.: US 2026/0165980 A1
(43) Pub. Date: Jun. 18, 2026

(54) LYMPHATIC ENDOTHELIAL CELL-SPECIFIC LIPID NANOPARTICLE AND USES THEREOF

(71) Applicants: GEORGIA TECH RESEARCH CORPORATION, Atlanta, GA (US); THE REGENTS OF THE UNIVERSITY OF COLORADO, A BODY CORPORATE, Denver, CO (US)

(72) Inventors: Eleftheria MICHALAKI, Atlanta, GA (US); James DAHLMAN, Atlanta, GA (US); Brandon DIXON, Atlanta, GA (US); Beth TAMBURINI, Denver, CO (US)

(21) Appl. No.: 19/122,926

(22) PCT Filed: Oct. 20, 2023
(86) PCT No.: PCT/US2023/077442

§ 371 Date: Apr. 21, 2025

Related U.S. Application Data
(60) Provisional application No. 63/380,429, filed on Oct. 21, 2022.

(51) Int. Cl. A61K 9/51; A61K 38/18; A61K 39/00; A61K 39/385; A61K 47/10; A61K 47/24; A61K 47/28

(52) U.S. Cl. CPC A61K 9/5123; A61K 38/1866; A61K 39/385; A61K 47/10; A61K 47/24; A61K 47/28; A61K 2039/53

(57) ABSTRACT

The present disclosure relates to lymphatic endothelial cell (LEC)-specific lipid nanoparticle comprising a sterol, an ionizable lipid, a PEGylated lipid, and a phospholipid. Also disclosed herein are compositions comprising the LEC-specific lipid nanoparticle and a therapeutic agent. The present disclosure further relates to methods of delivering an agent to a lymphatic system in a subject, comprising administering to the subject an effective amount of the composition of the LEC-specific lipid nanoparticle and therapeutic agent.

### CROSS-REFERENCE TO RELATED APPLICATIONS

This application claims priority to U.S. Provisional Patent Application No. 63/380,429, filed Oct. 21, 2022, which is incorporated herein by reference in its entirety.

### STATEMENT REGARDING FEDERALLY SPONSORED RESEARCH

This invention was made with government support under grant no. W81XWH-18-1-0506 awarded by U.S. Army Medical Research Acquisition Activity and grant no. 829544 awarded by the American Heart Association. The government has certain rights in the invention.

### FIELD

The present disclosure relates to lymphatic endothelial cell-specific nanoparticles and uses thereof.

### BACKGROUND

There is increasing evidence demonstrating the contribution of the lymphatic system to human diseases ranging from obesity and cardiovascular diseases to cancer and neurological disorders. Given the established role of the lymphatic system in multiple pathophysiological conditions, such as atherosclerosis and myocardial infarction, understanding how to exploit its properties of cargo transport can be a promising avenue toward new efficient therapies.

Advances in means of targeted therapy have shown promise for treating diseases that conventional therapies have been unable to resolve. For example, utilizing nanomedicine for targeted drug delivery serves to improve the effective dose at the target tissue while avoiding off-target effects. Therefore, what is needed are compositions and methods for targeted drug delivery to lymphatic system, and more specifically to lymphatic endothelial cells.

### SUMMARY

Disclosed herein are lymphatic endothelial cell-specific lipid nanoparticles comprising a sterol, an ionizable lipid, a PEGylated lipid, and a phospholipid.

In some embodiments, the lymphatic endothelial cell-specific lipid nanoparticles further include an alcohol. In some embodiments, the alcohol comprises ethanol.

In some embodiments, the phospholipid comprises 1,2-dioleoyl-sn-glycero-3-phosphoethanolamine (DOPE), 1-palmitoyl-2-oleoyl-sn-glycero-3-phosphoethanolamine (POPE), 1,2-distearoyl-sn-glycero-3-phosphocholine (DSPC), 1-stearoyl-2-oleoyl-sn-glycero-3-phosphoethanolamine (SOPE), DPPC (1,2-dipalmitoyl-sn-glycero-3-phosphocholine), 1,2-dioleyl-sn-glycero-3-phosphotidylcholine (DOPC), 1,2-dipalmitoyl-sn-glycero-3-phosphoethanolamine (DPPE), 1,2-dimyristoyl-sn-glycero-3-phosphoethanolamine (DMPE), 1,2-dioleoyl-5/7-glycero-3-phospho-(1′-rac-glycerol) (DOPG), dioctadecylammonium bromide (DDAB), biotinylated DOPE, or combinations thereof.

In some embodiments, the phospholipid is 2-dioleoyl-sn-glycero-3-phosphoethanolamine (DOPE)

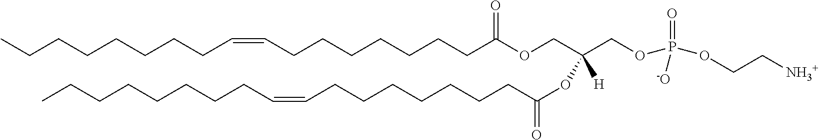

1,2-distearoyl-sn-glycero-3-phosphocholine (DSPC)

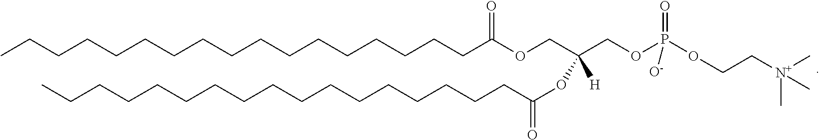 [SMILES: CCCCCCCCCCCCCCCCCC(=O)OC[C@H](COP(=O)([O-])OCC[N+](C)(C)C)OC(=O)CCCCCCCCCCCCCCCCC]

In some embodiments, the sterol comprises a cholesterol-based lipid. In some embodiments, the sterol comprises cholesterol. In some embodiments, the sterol comprises 20α-OH cholesterol.

In some embodiments, the ionizable lipid comprises

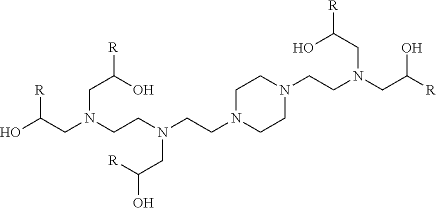

wherein each R is independently a substituted or unsubstituted C₈-C₁₈ alkyl. In some embodiments, each R is independently an unsubstituted C₈-C₁₈ alkyl. In some embodiments, each R is independently an unsubstituted C₁₂-C₁₃ alkyl.

In some embodiments, the ionizable lipid comprises

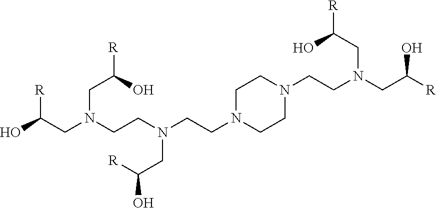

wherein each R is independently a substituted or unsubstituted C₈-C₁₈ alkyl. In some embodiments, each R is independently an unsubstituted C₈-C₁₈ alkyl. In some embodiments, each R is independently an unsubstituted C₁₂-C₁₃ alkyl.

In some embodiments, the ionizable lipid comprises

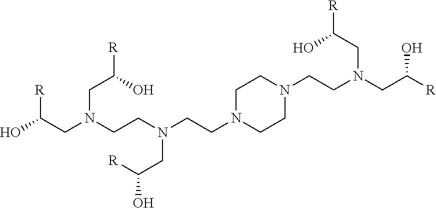

wherein each R is independently a substituted or unsubstituted C₈-C₁₈ alkyl. In some embodiments, each R is independently an unsubstituted C₈-C₁₈ alkyl. In some embodiments, each R is independently an unsubstituted C₁₂-C₁₃ alkyl.

In some embodiments, the ionizable lipid is cKK-E12:

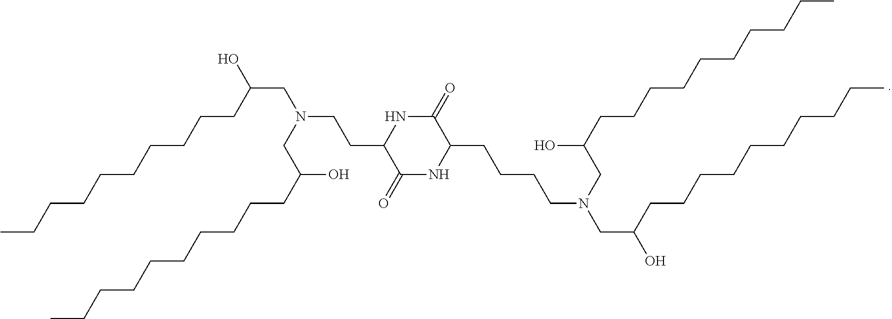 [SMILES: CCCCCCCCCCC(O)CN(CCCCC1NC(=O)C(CCN(CC(O)CCCCCCCCCC)CC(O)CCCCCCCCCC)NC1=O)CC(O)CCCCCCCCCC]

In some embodiments, the PEGylated lipid is C14PEG2000:

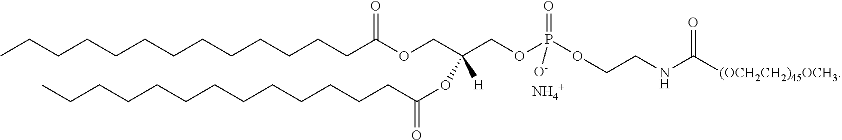

In some embodiments, the lymphatic endothelial cell-specific lipid nanoparticle comprises from 30% to 50% v/v alcohol, from 12% to 25% v/v of the ionizable lipid, from 5% to 18% v/v of the sterol, from 5% to 8% v/v of the PEGylated lipid, and from 5% to 35% v/v of the phospholipid.

In some embodiments, the lymphatic endothelial cell-specific lipid nanoparticle comprises from 35% to 40% v/v alcohol, from 15% to 20% v/v of the ionizable lipid, from 5% to 10% v/v of the sterol, from 5% to 8% v/v of the PEGylated lipid, and from 25% to 35% v/v of the phospholipid.

In some embodiments, the lymphatic endothelial cell-specific lipid nanoparticle comprises from 40% to 45% v/v alcohol, from 20% to 25% v/v of the ionizable lipid, from 10% to 15% v/v of the sterol, from 5% to 8% v/v of the PEGylated lipid, and from 5% to 10% v/v of the phospholipid.

In some embodiments, the lymphatic endothelial cell-specific lipid nanoparticle comprises 44.88% v/v ethanol, 24.88% v/v cKK-E12, 13.56% v/v cholesterol, 6.76% v/v C14PEG2000, and 9.92% v/v DSPC of the weight of the lymphatic endothelial cell-specific lipid nanoparticle.

In some embodiments, the lymphatic endothelial cell-specific lipid nanoparticle comprises 37.04% v/v ethanol, 17.04% v/v cKK-E12, 6.84% v/v cholesterol, 6.6% v/v C14PEG2000, and 32.48% v/v DOPE of the weight of the lymphatic endothelial cell-specific lipid nanoparticle.

In some embodiments, the lymphatic endothelial cell-specific lipid nanoparticle comprises 43.96% v/v ethanol, 23.96% v/v cKK-E12, 5.35% v/v cholesterol, 6.18% v/v C14PEG2000, and 20.55% v/v DOPE of the weight of the lymphatic endothelial cell-specific lipid nanoparticle.

In some embodiments, a molar ratio of the ionizable lipid to the other components is from 25% to 60% (e.g., from 25% to 55%, from 25% to 50%, from 25% to 45%, from 25% to 40%, from 25% to 35%, from 25% to 30%, from 30% to 60%, from 35% to 60%, from 40% to 60%, from 45% to 60%, from 50% to 60%). In some embodiments, a molar ratio of the sterol to the other components is from 15% to 50% (e.g., from 15% to 45%, from 15% to 40%, from 15% to 35%, from 15% to 30%, from 20% to 50%, from 25% to 50%, from 30% to 50%, from 35% to 50%, from 40% to 50%). In some embodiments, a molar ratio of the PEGylated lipid to the other components is from 8% to 45% (e.g., from 8% to 38%, from 8% to 33%, from 8% to 28%, from 8% to 23%, from 8% to 18%, from 8% to 13%, from 13% to 45%, from 18% to 45%, from 23% to 45%, from 28% to 45%, and from 33% to 45%). In some embodiments, a molar ratio of the phospholipid to the other components is from 0.1% to 8% (e.g., from 0.1% to 6.1%, from 0.1% to 5.1%, from 0.1% to 4.1%, from 0.1% to 3.1%, from 0.1% to 2.1%, from 0.1% to 1.1%, from 1.1% to 8%, from 2.1% to 8%, from 3.1% to 8%, from 4.1% to 8%, and from 5.1% to 8%).

In some embodiments, the lymphatic endothelial cell-specific lipid nanoparticle has an average particle size from about 50 nm to about 150 nm. In some embodiments, the lymphatic endothelial cell-specific lipid nanoparticle has an average particle size from about 50 nm to about 100 nm.

Also disclosed herein is a composition comprising: the lymphatic endothelial cell-specific lipid nanoparticle disclosed herein and a therapeutic agent. In some embodiments, the therapeutic agent comprises a nucleic acid, a peptide, or a small molecule. In some embodiments, the nucleic acid is a DNA or RNA. In some embodiments, the nucleic acid, peptide, or small molecule comprises a VEGF protein, a nucleotide encoding a VEGF protein, or a fragment thereof. In some embodiments, the nucleic acid, a peptide, or a small molecule comprises a nucleotide encoding a VEGF-C protein.

In various aspects, disclosed herein is a method of delivering an agent to a lymphatic system in a subject, comprising administering to the subject an effective amount of any of the compositions described herein.

Also described herein is a method of treating a lymphatics-associated disorder in a subject, comprising administering to the subject a therapeutically effective amount of any of the lymphatic endothelial cell-specific lipid nanoparticles or any of the compositions provided herewith.

In some embodiments, the lymphatics-associated disorder is a cardiovascular disease, lymphatic vascular injury, or brain injury.

In some embodiments, the lymphatic endothelial cell-specific lipid nanoparticle does not promote cancer metastasis.

The disclosure further provides a method of enhancing lymphangiogenesis in a subject, comprising administering to the subject a therapeutically effective amount of any of the lymphatic endothelial cell-specific lipid nanoparticles or any of the compositions provided herewith.

In some embodiments, the subject has a lymphatics-associated disorder. In some embodiments, the lymphatics-associated disorder is a cardiovascular disease, lymphatic vascular injury, or brain injury. In some embodiments, the lymphatic endothelial cell-specific lipid nanoparticle does not promote cancer metastasis.

An mRNA vaccine comprising any of the described lymphatic endothelial cell-specific lipid nanoparticles and an mRNA disposed therewithin. In some embodiments, the mRNA vaccine further includes a pharmaceutically acceptable carrier.

Also described herein is a method of preventing or treating a disorder or an infection in a subject, comprising administering to the subject a therapeutically effective amount of any of the mRNA vaccines described herein.

In some embodiments, the infection is a viral infection, a bacterial infection, a parasitic infection, or a yeast infection. In some embodiments, the disorder is a cancer.

In various embodiments, described herein is a pharmaceutical composition comprising any of the lymphatic endothelial cell-specific lipid nanoparticles or any of the compositions provided herewith and a pharmaceutically acceptable carrier.

### DESCRIPTION OF DRAWINGS

FIGS. 1A-1D shows characterization of LNP7 tested with FIND. (FIG. 1A) LNP libraries with aVHH mRNA and unique DNA barcode (BC) are administered to C57Bl6/J mice. aVHH aVHH+ cells are isolated by FACS and DNA barcodes are sequenced. (FIG. 1B) Formulation compounds of LNP7. (FIG. 1C) Composition and (FIG. 1D) Characterization of LNP7 discovered by FIND.

FIGS. 2A-2B show an LNP based platform for functional mRNA delivery of VEGF C targeting the lymphatic system. FIG. 2A. Percentage of aVHH cells (after gating for Live/Dead, CD31++/PDPN++, and relative to saline) from ALN, BLN, and PLN that have successfully taken up LNP1 and LNP7. Solid line above plots indicates a pairwise comparison for significance using a two-way ANOVA with p<0.001 (***). FIG. 2B. Percentage of VEGF C cells (after gating for Live/Dead and CD31++/PDPN++) in saline and LNP7 conditions (cells pooled from ALN, BLN, and PLN).

FIGS. 3A-3F show immunofluorescence micrographs of popliteal lymphatic vessel segments in Saline and LNP7 injected Ai14 mice for: (FIG. 3A) & (FIG. 3D) DAPI, (FIG. 3B) & (FIG. 3E) PDPN, and (FIG. 3C) & (FIG. 3F) tdTom LNP7. (40× objective; Scale bar=100 m.) The contrast was enhanced post-acquisition equally in all image panels for ease of viewing.

FIGS. 4A-4B show characterization of LNPs libraries tested with FIND. (FIG. 4A) Composition and (FIG. 4B) Characterization of lead LNPs discovered by FIND, i.e., LNP1, LNP72, LNP7 and LNP11.

FIG. 5 shows that LNP4 leads to higher LEC-specific uptake in ALNs compared to other lead LNP candidates.

FIG. 6 shows that LNP7 leads to higher LEC-specific uptake in BLNs compared to other lead LNP candidates.

FIG. 7 shows that LNP7 leads to higher LEC-specific uptake in PLNs compared to other lead LNP candidates.

FIG. 8 shows that LNP7 leads to higher LEC-specific uptake in PLNs compared to LNP1.

FIGS. 9A-9E show (FIG. 9A) Composition of the LNP libraries tested. (FIG. 9B) Each of the compounds was formulated using 14 molar ratios. (FIG. 9C) Of the 150 LNPs that were formulated, 99 passed the quality control (QC) criteria, with a diameter less than 200 nm as well as a stable autocorrelation curve. (FIG. 9D) Hydrodynamic diameters and (FIG. 9E) Polydispersity indexes (PDI) of all administered LNPs; the diameter of the LNP pooled control is within the range of the LNPs composing the pool.

FIGS. 10A-10D show (FIG. 10A) Formulation compounds, composition, hydrodynamic diameter (nm), and PDI of lead LEC-specific LNPs. Percentage of aVHH+ LECs (after gating for Live/Dead, CD31⁺/PDPN⁺) from (FIG. 10B) axillary LNs (ALN), (FIG. 10C) brachial LNs (BLN), and (FIG. 10D) popliteal LNs (PLN) that have successfully taken up saline, LNP1, LNP2, LNP3, LNP4, LNP7, and LNP11. Each data point corresponds to an independent experiment and error bars represent the standard error of the mean. Asterisks indicate a comparison with the corresponding administration using a one-way ANOVA and robust regression and outlier removal (ROUT) method to identify and remove outliers with p<0.05 (*), p<0.01 (**), p<0.001 (***), and p<0.0001 (****).

FIGS. 11A-11C show (FIG. 11A) formulation compounds, composition, hydrodynamic diameter (nm), PDI, encapsulation efficiency (%), total mRNA concentration (μg/mL), encapsulated mRNA concentration (μg/mL) of MC3 and LNP7. (FIG. 111B) Percentage of aVHH⁺ LECs (after gating for Live/Dead, CD45⁻/CD31⁺/PDPN⁺) from popliteal LNs and LVs that have successfully taken up saline, free aVHH, MC3 (standard positive control LNP) and LNP7. (FIG. 11C) Percentage of aVHH⁺ uptake of LNP7 by different cell types, namely LECs, BECs, VECs, Macrophages, Monocytes, cDC2, cDC1, DNs, and FRCs in PLN Inj. Each data point corresponds to an independent experiment and error bars represent the standard error of the mean. (FIG. 111B) Asterisks indicate a comparison with the corresponding administration using a two-way ANOVA with Tukey's multiple comparisons test with p<0.05 (*) and p<0.0001 (****). (FIG. 11C) Solid lines above plots indicate a pairwise comparison for significance using a one-way ANOVA with p<0.05 (*) and p<0.01 (**).

FIGS. 12A-12D show VEGFC mRNA administration significantly increases the frequency 7-days after mouse tail lymphedema surgery in vivo. (FIG. 12A) Frequency, (FIG. 12B) Amplitude, (FIG. 12C) Packet Integral, and (FIG. 12D) Packet Transport for empty, 0.04 μg, 0.2 μg, 1 μg, and 5 μg 7-days post-surgery. Each data point corresponds to an independent experiment (Nempty=6, N0.04 μg=5, N0.2 μg=5, N01 μg=4, and N5 μg=8), and error bars indicate the corresponding standard error of the mean. Solid lines above the plots indicate a pairwise comparison for significance using one-way ANOVA with Tukey's multiple comparisons test with p<0.05 (*), p<0.01 (**), and p<0.001 (***).

FIGS. 13A-13B show that VEGFC mRNA overexpression significantly increased the PDPN/EdU colocalization in the wound site after mouse tail lymphedema surgery in vivo. (FIG. 13A) Representative images of merged, DAPI, PDPN, and EdU in tail sections for empty and g in wound and distal sites 7 days post-surgery. (20× objective; Scale bar=50 μm.) The contrast was enhanced post-acquisition equally in all image panels for ease of viewing. (FIG. 13B) Pearson's coefficient for empty and 5 μg 7-days post-surgery in the wound and distal sites. Each data point corresponds to the average of each independent experiment (NEmpty=6 and N5 μg=7), and error bars indicate the corresponding standard error of the mean. Solid lines above plots indicate a pairwise comparison for significance using a nested one-way ANOVA with Tukey's multiple comparisons test with p<0.05 (*).

FIGS. 14A-14F show that LNP7-loaded with VEGFC mRNA significantly increased the frequency 14-days after mouse tail lymphedema surgery in vivo. (FIG. 14A) Frequency, (FIG. 14B) Amplitude, (FIG. 14C) Packet Integral, (FIG. 14D) Packet Transport, (FIG. 14E) Absolute Tail Volume Change, and (FIG. 14F) Normalized Tail Volume for empty, MC3/VEGFC (5 μg), and LNP7/VEGFC (5 μg) 14-days post-surgery. Each data point corresponds to an independent experiment (N_{Empty}=11, N_{MC3/VEGFC}=13, and N_{LNP7/VEGFC}=10), and error bars indicate the corresponding standard error of the mean. Solid lines above the plots indicate a pairwise comparison for significance using Mixed-effects analysis with Tukey's multiple comparisons test and robust regression and outlier removal (ROUT) method to identify and remove outliers with p<0.05 (*), p<0.01 (**), and p<0.001 (***).

FIG. 15 shows Steps for LNP formulation and quality assessment. The LNP recipe is determined, and the corresponding citrate and lipid phases are combined in the microfluidic device for LNP formulation. The quality of the formulated LNP is evaluated using dynamic light scattering (DLS) to measure the diameter and polydispersity index (PDI), nanodrop to measure the mRNA concentration, and encapsulation assay to quantify the corresponding encapsulation efficiency.

FIG. 16 shows that LNP7 can carry multiple mRNA cargos. Hydrodynamic diameter (nm), PDI, encapsulation efficiency (%), total mRNA concentration (μg/mL), encapsulated mRNA concentration (μg/mL) of MC3 and LNP7 with different mRNA cargos, i.e., aVHH mRNA and VEGFC mRNA.

FIG. 17 shows the effect of the route of administration on LNP delivery. Percentage of aVHH⁺ cells (after gating for Live/Dead, CD31⁺/PDPN⁺, and relative to saline) in PLNs that have successfully taken up the LNP cargo after intravenous (IV) and intradermal (ID) injections. Each data point corresponds to an independent experiment and error bars represent the standard error of the mean. Solid line above plots indicates a pairwise comparison for significance using an unpaired t-test with p<0.05 (*).

FIGS. 18A-18I show that LNP uptake by various cell populations that reside in LNs. Percentage of aVHH⁺ LECs (after gating for Live/Dead, CD45⁻/CD31⁺/PDPN⁺) from popliteal LNs that have successfully taken up saline, free aVHH, MC3 (standard positive control LNP) and LNP7. Cell populations studied include: LECs (FIG. 18A), BECs (FIG. 18B), VECs (FIG. 18C), Macrophages (FIG. 18D), Monocytes (FIG. 18E), cDC2 (FIG. 18F), cDC1 (FIG. 18G), DNs (FIG. 18H), FRCs (FIG. 18I). Each data point corresponds to an independent experiment and error bars represent the standard error of the mean. Solid lines above plots indicate a pairwise comparison for significance using a two-way ANOVA with Tukey's multiple comparisons test with p<0.05 (*), p<0.01 (**), p<0.001 (***), and p<0.0001 (****).

FIGS. 19A-19B show that LEC-specific LNP (i.e., LNP7) is not toxic. (FIG. 19A) Immunofluorescence micrographs of human LECs in EBM-, DMSO-, and LNP7-treated cells for merged, DAPI, and Live/Dead. (20× objective; Scale bar=50 m.) The contrast was enhanced post-acquisition equally in all image panels for ease of viewing. (FIG. 19B) Quantification of the L/D staining and alamarBlue fluorescence intensity. Each data point corresponds to an independent experiment and error bars represent the standard error of the mean. Solid lines above plots indicate a pairwise comparison for significance using a one-way ANOVA with p<0.05 (*) and p<0.01 (**). Error bars represent the standard error of the mean. Solid lines above plots indicate a pairwise comparison for significance using a two-way ANOVA with Tukey's multiple comparisons test with p<0.05 (*), p<0.01 (**), p<0.001 (***), and p<0.0001 (****). Asterisks indicate comparison with the corresponding treatment using a one-way ANOVA with Tukey's multiple comparisons test and robust regression and outlier removal (ROUT) method to identify and remove outliers with p<0.01 (**), p<0.001 (***) and p<0.0001 (****).

FIG. 20 shows IHC controls in tail LVs. Immunofluorescence micrographs of tail LV segments for sham mice injected with saline (negative control), sham mice injected with EdU, and lymphedema mice injected with EdU 7 days post-surgery for merged, DAPI, PDPN, and EdU. (20× objective; Scale bar=50 m.) The contrast was enhanced post-acquisition equally in all image panels for ease of viewing.

FIG. 21 shows representative gating strategies for FACS for various cell populations present in LNs. Identifying the following cell populations via appropriate gating as seen above; LECs: CD45⁻/CD31⁺/PDPN⁺, VECs: CD45⁻/CD31⁺/PDPN⁻/CD54⁺, BECs: CD45⁻/CD31⁺/PDPN⁻/CD309⁺, FRCs: CD45⁻/CD31⁻/PDPN⁺, DNs: CD45⁻/CD31⁻/PDPN⁻, Monocytes: CD45⁺/CD11b⁺/CD64⁺/F4-80⁻, Macrophages: CD45⁺/CD11b⁺/CD64⁻/F4-80⁺, cDC1: CD45⁺/CD11c⁺/MHCII⁺/CD11b⁺, and cDC2: CD45⁺/CD11c⁺/MHCII⁺/CD11b⁺.

FIG. 22 shows representative gating strategies for FACS for LECs in LVs. Identifying the LEC population via appropriate gating (CD31⁺/PDPN⁺) as seen above.

FIGS. 23A-23C show (FIG. 23A) an overview of experimental design of Example 3 where a vaccine was LNP encapsulated mRNA encoding SIINFEKL-GFP (2 ug RNA per injection site) or ovalbumin, polyL:C, anti-CD40. The vaccine was administered subcutaneously (S.C) in the mouse footpad. (FIG. 23B) At indicated time-points after vaccination with LNP7 as the lipid coat, draining LNs were removed and digested prior to staining cells for markers of lymph node stromal cells (LNSC) and dendritic cells (DC)/monocytes. GFP was only detected in DC populations (both migratory and LN resident) and LNSC populations indicated. (FIG. 23C) At day 17 post vaccination with indicated LNPs that encapsulated the SIINFEKL-GFP mRNA, mice were euthanized and LNs removed and processed for detection of transferred violet proliferation dye VPD labeled OT1 CD8 T cells. Shown are dilution peaks of the VPD label with ova/polyL:C/aCD40 as a positive control of a vaccine that induces antigen archiving.

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

The term “biocompatible” generally refers to a material and any metabolites or degradation products thereof that are generally non-toxic to the recipient and do not cause significant adverse effects to the subject.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

A “control” is an alternative subject or sample used in an experiment for comparison purposes. A control can be “positive” or “negative.”

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence.

The term “increased” or “increase” as used herein generally means an increase by a statically significant amount; for example, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “nanoparticle” as used herein refers to a particle or structure which typically ranges from about 1 nm to about 1000 nm in size, for example, from about 50 nm to about 500 nm size, from about 50 nm to about 350 nm size, or from about 50 nm to about 200 nm size.

The term “nucleic acid” as used herein means a polymer composed of nucleotides, e.g., deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides. (Used together with “polynucleotide” and “polypeptide”.)

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ).

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The term “sterol” refers to a subgroup of steroids also known as steroid alcohols. Sterols are usually divided into two classes: (1) plant sterols also known as “phytosterols”; and (2) animal sterols also known as “zoosterols.” Examples of sterols include, for example, campesterol, sitosterol, stigmasterol, ergosterol and cholesterol.

The term “target” refers to a molecule that has an affinity for a given probe. Targets may be naturally-occurring or man-made molecules. Also, they can be employed in their unaltered state or as aggregates with other species.

The terms “treat,” “treating,” “treatment,” and grammatical variations thereof as used herein, include partially or completely delaying, alleviating, mitigating or reducing the intensity of one or more attendant symptoms of cancer or condition and/or alleviating, mitigating or impeding one or more symptoms of cancer. Treatments according to the invention may be applied preventively, prophylactically, palliatively or remedially. Prophylactic treatments are administered to a subject prior to onset (e.g., before obvious signs of cancer), during early onset (e.g., upon initial signs and symptoms of cancer), after an established development of cancer, or during prevention or mitigation of cancer relapse. Prophylactic administration can occur for several minutes to months prior to the manifestation of a disease.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Lymphatic disorders are conditions in which there is a deviation from or interruption of the normal structure or function of the lymph or lymph vessels. Disorders of the lymphatic system affect millions of people and include, but are not limited to: lymphedema, the most severe lymphatic disorder in which patients are often susceptible to serious life-threatening cellulite infections that if uncontrolled can spread systemically or lead to amputation (both primary and secondary forms, including, but not limited to, lymphangiomatosis, lymphangioleiomyomatosis and other mixed vascular/lymphatic malformation syndromes or conditions, such as Turner-Weber and Klippel Trenauney Syndrome and those that result from filariasis, trauma, infection or surgeries of the breast, prostate, uterus, cervix, abdomen, as well as orthopedic, cosmetic (liposuction) and other surgeries, malignant melanoma, and treatments used for both Hodgkin's and non-Hodgkin's lymphoma, radiation therapy, sports injuries, tattooing, diabetes, obesity and any physical insult to the lymphatic pathways); lymphangiogenesis (or the process of growing new lymphatic structures); the inability to control infections such as that associated with HIV/AIDS; the inability to deliver antibiotic and anti-viral medication to infected tissues and organs; inflammatory and auto-immune diseases, such as but not limited to, rheumatoid arthritis and systemic lupus erythematosis, scleroderma, Wegener's granulomatosis; lymphatic insufficiency of the internal organs; impairment of lymphatic development in the intestines, for example, leads to malabsorption, ascites (collections of fat-laden lymph within the abdominal cavity), underdevelopment from malnutrition, immune malfunction, and premature death; and pulmonary lymphangiectasia, cystic hygromas and lymphangiomas that may lead to impaired vision, swallowing and breathing. Collectively, such diseases and disorders are referred to herein as “lymphatic-associated disorders” or “lymphatic disorders.”

As used herein, the term “lymphangiogenesis” refers to the growth of new lymphatic vessels. Lymphangiogenesis can occur at a variety of sites in the subject. Lymphangiogenesis can be involved in a variety of pathological or disease conditions including, for example, neoplasm metastasis, edema, rheumatoid arthritis, psoriasis, lymphangiomatosis and impaired wound healing.

The term “mRNA vaccine” as used herein refers to a vaccine that induces an immune response by artificially replicating some of the genes of an antigen and then administering it. These mRNA vaccines have various advantages compared to the existing protein vaccines. First of all, there is no need to directly handle dangerous pathogens because they can be synthesized only with the genetic information of the pure target antigen. Even if it does, there is no concern that it will show any particular toxicity, and it has various advantages such as being able to rapidly develop vaccines against suddenly occurring infectious diseases or various mutations due to the simplicity of its composition only with mRNA.

### Chemical Definitions

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety. The prefix Cn-Cm preceding a group or moiety indicates, in each case, the possible number of carbon atoms in the group or moiety that follows.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acetal, acyl, alkyl, alkoxy, alkenyl, alkynyl, aryl, heteroaryl, aldehyde, amino, cyano, carboxylic acid, ester, ether, carbonate ester, carbamate ester, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkyl alcohol” is used in another, it is not meant to imply that the term “alkyl” does not also refer to specific terms such as “alkyl alcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-C₁₈, C₂-C₁₆, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some embodiments, aryl groups include C₆-C₁₀ aryl groups. Examples of aryl groups include, but are not limited to, benzene, phenyl, biphenyl, naphthyl, tetrahydronaphthyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems (e.g., monocyclic, bicyclic, tricyclic, polycyclic, etc.) that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a shorthand notation for C═O.

The term “acetal” as used herein is represented by the formula (Z¹Z²)C(═OZ³)(═OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

The term “alkyl alcohol” as used herein is represented by the formula Z¹OH, where Z¹ can be an alkyl, alkenyl, alkynyl, aryl, heteroaryl, cycloalkyl, cycloalkenyl, heterocycloalkyl, or heterocycloalkenyl group described above. Examples of alkyl-alcohols include methanol, ethanol, 1-propanol, 2-propanol, 1-butanol, 2-butanol, tert-butyl alcohol, and the like.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula C(O)H. Throughout this specification “C(O)” is a shorthand notation for C═O.

The terms “amine” or “amino” as used herein are represented by the formula NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula C(O)NZ¹Z², where Z¹ and Z² can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “anhydride” as used herein is represented by the formula Z¹C(O)OC(O)Z² where Z¹ and Z², independently, can be an alkyl, alkenyl, alkynyl, aryl, heteroaryl, cycloalkyl, cycloalkenyl, heterocycloalkyl, or heterocycloalkenyl group described above.

The term “cyclic anhydride” as used herein is represented by the formula:

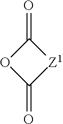

where Z¹ can be an alkyl, alkenyl, alkynyl, aryl, heteroaryl, cycloalkyl, cycloalkenyl, heterocycloalkyl, or heterocycloalkenyl group described above.

The term “azide” as used herein is represented by the formula —N═N═N.

The term “carboxylic acid” as used herein is represented by the formula —C(O)OH.

A “carboxylate” or “carboxyl” group as used herein is represented by the formula —C(O)O⁻.

A “carbonate ester” group as used herein is represented by the formula Z¹OC(O)OZ².

The term “cyano” as used herein is represented by the formula —CN.

The term “ester” as used herein is represented by the formula —Z¹OC(O)Z² or Z¹C(O)OZ², where Z¹ and Z² can independently be be an alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “epoxy” or “epoxide” as used herein refers to a cyclic ether with a three atom ring and can represented by the formula:

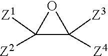

where Z¹, Z², Z³, and Z⁴ can be, independently, an alkyl, alkenyl, alkynyl, aryl, heteroaryl, cycloalkyl, cycloalkenyl, heterocycloalkyl, or heterocycloalkenyl group described above

The term “ketone” as used herein is represented by the formula Z¹C(O)Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula OH.

The term “nitro” as used herein is represented by the formula N02.

The term “phosphonyl” is used herein to refer to the phospho-oxo group represented by the formula —P(O)(OZ¹)₂, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “silyl” as used herein is represented by the formula —SiZ¹Z²Z³, where Z¹, Z², and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above.

The term “sulfonyl” or “sulfone” is used herein to refer to the sulfo-oxo group represented by the formula S(O)₂Z¹, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein is comprises the formula S.

The term “thiol” as used herein is represented by the formula SH.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

### Nanoparticles and Methods

Disclosed herein are techniques and compositions for cargo delivery into the lymphatic system, such as lymphatic endothelial cells (LECs). The development of a versatile tool targeting lymphatic endothelial cells (LECs) can revolutionize targeted therapy in a variety of disease processes associated with the lymphatic system ranging from cardiovascular diseases to cancer and brain injuries. Targeted therapy utilizing a LEC-specific LNP disclosed herein is the first attempt toward efficient mRNA delivery in LECs and its corresponding use as a therapeutic modality.

In some aspect, disclosed herein is a lymphatics-specific (e.g., specifically targeting lymphatic endothelial cells (LECs)) lipid nanoparticle comprising one or more of ethanol, cholesterol, an ionizable lipid, a PEGylated lipid, and a phospholipid. In some aspect, disclosed herein is a LEC-specific lipid nanoparticle comprising one or more of ethanol, cholesterol, an ionizable lipid, a PEGylated lipid, and a phospholipid. In some aspect, disclosed herein is a LEC-specific lipid nanoparticle comprising ethanol (e.g., from 20% v/v to 50% v/v nanoparticle weight), cholesterol (e.g., from 5% v/v to 20% v/v nanoparticle weight), an ionizable lipid (e.g., from 10% v/v to 30% v/v nanoparticle weight), a PEGylated lipid (e.g., from 5% v/v to 10% v/v nanoparticle weight), and a phospholipid (e.g., from 5% v/v to 50% v/v nanoparticle weight). In some aspect, disclosed herein is a LEC-specific lipid nanoparticle comprising cholesterol, an ionizable lipid, a PEGylated lipid, and a phospholipid.

The lipid component of a lipid nanoparticle composition can include one or more molecules comprising polyethylene glycol, such as PEG or PEG-modified lipids. Such species are also referred to as “PEGylated lipids.” A PEGylated lipid (also known as a PEG lipid or a PEG-modified lipid) is a lipid modified with polyethylene glycol. A PEGylated lipid can be selected from the non-limiting group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEGylated lipid may be PEG-c-DOMG, PEG-DMG, PEG-DLPE, PEG-DMPE, PEG-DPPC, PEG-DSG, or a PEG-DSPE lipid.

In some embodiments, the phospholipid comprises 1,2-dioleoyl-sn-glycero-3-phosphoethanolamine (DOPE), 1-palmitoyl-2-oleoyl-sn-glycero-3-phosphoethanolamine (POPE), 1,2-distearoyl-sn-glycero-3-phosphocholine (DSPC), 1-stearoyl-2-oleoyl-sn-glycero-3-phosphoethanolamine (SOPE), DPPC (1,2-dipalmitoyl-sn-glycero-3-phosphocholine), 1,2-dioleyl-sn-glycero-3-phosphotidylcholine (DOPC), 1,2-dipalmitoyl-sn-glycero-3-phosphoethanolamine (DPPE), 1,2-dimyristoyl-sn-glycero-3-phosphoethanolamine (DMPE), 1,2-dioleoyl-5/7-glycero-3-phospho-(1′-rac-glycerol) (DOPG), dioctadecylammonium bromide (DDAB), biotinylated DOPE, or combinations thereof.

In some embodiments, the phospholipid is 2-dioleoyl-sn-glycero-3-phosphoethanolamine (DOPE)

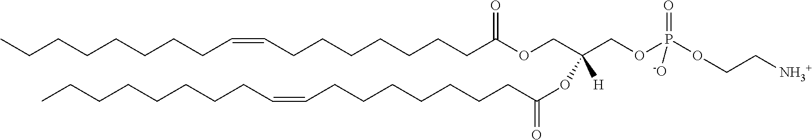

or
1,2-distearoyl-sn-glycero-3-phosphocholine (DSPC)

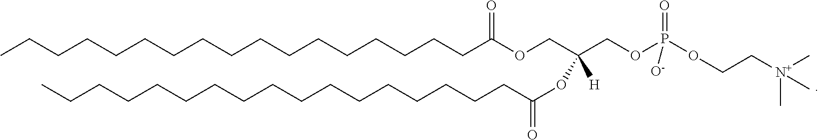 [SMILES: CCCCCCCCCCCCCCCCCC(=O)OC[C@H](COP(=O)([O-])OCC[N+](C)(C)C)OC(=O)CCCCCCCCCCCCCCCCC]

In some embodiments, the sterol comprises one or more cholesterol-based lipid. In some embodiments, the one or more cholesterol-based lipids are selected from cholesterol, PEGylated cholesterol, DC-Choi (N,N-dimethyl-N-ethylcarboxamidocholesterol), 1,4-bis(3-N-oleylamino-propyl)piperazine, or combinations thereof. In some embodiments, the sterol comprises cholesterol. In some embodiments, the sterol comprises 20α-OH cholesterol.

In some embodiments, the ionizable lipid comprises

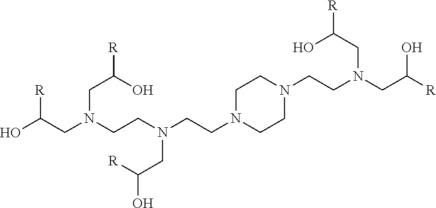

wherein each R is independently a substituted or unsubstituted C₈-C₁₈ alkyl. In some embodiments, each R is independently an unsubstituted C₈-C₁₈ alkyl. In some embodiments, each R is independently an unsubstituted C₁₂-C₁₃ alkyl.

In some embodiments, the ionizable lipid comprises

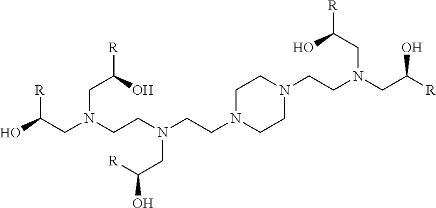

wherein each R is independently a substituted or unsubstituted C₈-C₁₈ alkyl. In some embodiments, each R is independently an unsubstituted C₈-C₁₈ alkyl. In some embodiments, each R is independently an unsubstituted C₁₂-C₁₃ alkyl.

In some embodiments, the ionizable lipid comprises

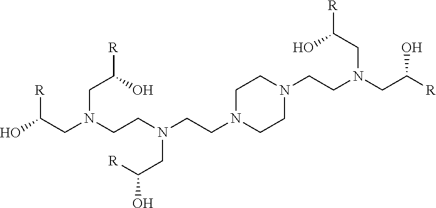

wherein each R is independently a substituted or unsubstituted C₈-C₁₈ alkyl. In some embodiments, each R is independently an unsubstituted C₈-C₁₈ alkyl. In some embodiments, each R is independently an unsubstituted C₁₂-C₁₃ alkyl.

In some embodiments, the ionizable lipid is cKK-E12:

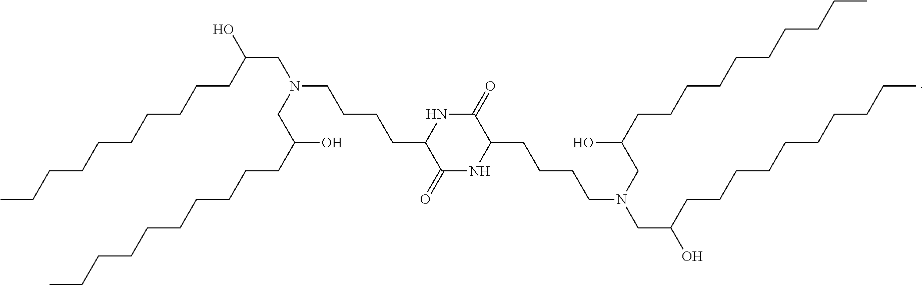 [SMILES: CCCCCCCCCCC(O)CN(CCCCC1NC(=O)C(CCCCN(CC(O)CCCCCCCCCC)CC(O)CCCCCCCCCC)NC1=O)CC(O)CCCCCCCCCC]

In some embodiments, the PEGylated lipid is C14PEG2000:

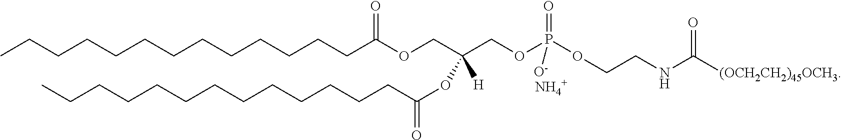

In some aspect, disclosed herein is a lymphatic endothelial cell-specific lipid nanoparticle comprising one or more of ethanol, cholesterol, cKK-E12, C14PEG2000, and DOPE. In some aspect, disclosed herein is a lymphatic endothelial cell-specific lipid nanoparticle comprising one or more of ethanol, cholesterol, cKK-E12, C14PEG2000, and (DSPC).

In some embodiments, the lymphatic endothelial cell-specific lipid nanoparticle comprises from 30% to 50% v/v alcohol, from 12% to 25% v/v of the ionizable lipid, from 5% to 18% v/v of the sterol, from 5% to 8% v/v of the PEGylated lipid, and from 5% to 35% v/v of the phospholipid.

In some embodiments, the lymphatic endothelial cell-specific lipid nanoparticle comprises from 35% to 40% v/v alcohol, from 15% to 20% v/v of the ionizable lipid, from 5% to 10% v/v of the sterol, from 5% to 8% v/v of the PEGylated lipid, and from 25% to 35% v/v of the phospholipid.

In some embodiments, the lymphatic endothelial cell-specific lipid nanoparticle comprises from 40% to 45% v/v alcohol, from 20% to 25% v/v of the ionizable lipid, from 10% to 15% v/v of the sterol, from 5% to 8% v/v of the PEGylated lipid, and from 5% to 10% v/v of the phospholipid.

In some embodiments, the lymphatic endothelial cell-specific lipid nanoparticle comprises 44.88% v/v ethanol, 24.88% v/v cKK-E12, 13.56% v/v cholesterol, 6.76% v/v C14PEG2000, and 9.92% v/v DSPC of the weight of the lymphatic endothelial cell-specific lipid nanoparticle.

In some embodiments, the lymphatic endothelial cell-specific lipid nanoparticle comprises 37.04% v/v ethanol, 17.04% v/v cKK-E12, 6.84% v/v cholesterol, 6.6% v/v C14PEG2000, and 32.48% v/v DOPE of the weight of the lymphatic endothelial cell-specific lipid nanoparticle.

In some embodiments, the lymphatic endothelial cell-specific lipid nanoparticle comprises 43.96% v/v ethanol, 23.96% v/v cKK-E12, 5.35% v/v cholesterol, 6.18% v/v C14PEG2000, and 20.55% v/v DOPE of the weight of the lymphatic endothelial cell-specific lipid nanoparticle.

In some embodiments, a molar ratio of the ionizable lipid to the other components is from 25% to 60% (e.g., from 25% to 55%, from 25% to 50%, from 25% to 45%, from 25% to 40%, from 25% to 35%, from 25% to 30%, from 30% to 60%, from 35% to 60%, from 40% to 60%, from 45% to 60%, from 50% to 60%). In some embodiments, a molar ratio of the sterol to the other components is from 15% to 50% (e.g., from 15% to 45%, from 15% to 40%, from 15% to 35%, from 15% to 30%, from 20% to 50%, from 25% to 50%, from 30% to 50%, from 35% to 50%, from 40% to 50%). In some embodiments, a molar ratio of the PEGylated lipid to the other components is from 8% to 45% (e.g., from 8% to 38%, from 8% to 33%, from 8% to 28%, from 8% to 23%, from 8% to 18%, from 8% to 13%, from 13% to 45%, from 18% to 45%, from 23% to 45%, from 28% to 45%, and from 33% to 45%). In some embodiments, a molar ratio of the phospholipid to the other components is from 0.1% to 8% (e.g., from 0.1% to 6.1%, from 0.1% to 5.1%, from 0.1% to 4.1%, from 0.1% to 3.1%, from 0.1% to 2.1%, from 0.1% to 1.1%, from 1.1% to 8%, from 2.1% to 8%, from 3.1% to 8%, from 4.1% to 8%, and from 5.1% to 8%).

In some embodiments, the lymphatic endothelial cell-specific lipid nanoparticle has an average particle size from about 1 nm to about 1000 nm. In some embodiments, the lymphatic endothelial cell-specific lipid nanoparticle has an average particle size less than, for example, about 1000 nm, about 950 nm, about 900 nm, about 850 nm, about 800 nm, about 750 nm, about 700 nm, about 650 nm, about 600 nm, about 550 nm, about 500 nm, about 450 nm, about 400 nm, about 350 nm, about 300 nm, about 290 nm, about 280 nm, about 270 nm, about 260 nm, about 250 nm, about 240 nm, about 230 nm, about 220 nm, about 210 nm, about 200 nm, about 190 nm, about 180 nm, about 170 nm, about 160 nm, about 150 nm, about 140 nm, about 130 nm, about 120 nm, about 110 nm, about 100 nm, about 90 nm, about 80 nm, about 70 nm, about 60 nm, about 50 nm, about 40 nm, about 30 nm, about 20 nm, or about 10 nm. In some embodiments, the nanoparticle has a an average particle size, for example, from about 20 nm to about 1000 nm, from about 20 nm to about 800 nm, from about 20 nm to about 700 nm, from about 30 nm to about 600 nm, from about 30 nm to about 500 nm, from about 40 nm to about 400 nm, from about 40 nm to about 300 nm, from about 40 nm to about 250 nm, from about 50 nm to about 250 nm, from about 50 nm to about 200 nm, from about 50 nm to about 150 nm, from about 60 nm to about 150 nm, from about 70 nm to about 150 nm, from about 80 nm to about 150 nm, from about 90 nm to about 150 nm, from about 100 nm to about 150 nm, from about 110 nm to about 150 nm, from about 120 nm to about 150 nm, from about 90 nm to about 140 nm, from about 90 nm to about 130 nm, from about 90 nm to about 120 nm, from 100 nm to about 140 nm, from about 100 nm to about 130 nm, from about 100 nm to about 120 nm, from about 100 nm to about 110 nm, from about 110 nm to about 120 nm, from about 110 nm to about 130 nm, from about 110 nm to about 140 nm, from about 90 nm to about 200 nm, from about 100 nm to about 195 nm, from about 110 nm to about 190 nm, from about 120 nm to about 185 nm, from about 130 nm to about 180 nm, from about 140 nm to about 175 nm, from 150 nm to 175 nm, or from about 150 nm to about 170 nm. The particles can have any shape but are generally spherical in shape. In some embodiments, the lymphatic endothelial cell-specific lipid nanoparticle has an average particle size from about 50 nm to about 150 nm. In some embodiments, lymphatic endothelial cell-specific lipid nanoparticle has an average particle size from about 50 nm to about 100 nm.

The lipid particles can have an average particle size. “Average particle size” and “mean particle size” are used interchangeably herein, and generally refer to the statistical mean particle size of the particles in a population of particles. For example, the average particle size for a plurality of particles with a substantially spherical shape can comprise the average diameter of the plurality of particles. For a particle with a substantially spherical shape, the diameter of a particle can refer, for example, to the hydrodynamic diameter. As used herein, the hydrodynamic diameter of a particle can refer to the largest linear distance between two points on the surface of the particle. Mean particle size can be measured using methods known in the art, such as evaluation by scanning electron microscopy, transmission electron microscopy, and/or dynamic light scattering.

With respect to particle size distribution characterization, a parameter used to define the size range of the lipid particles is called the “polydispersity index” (PDI). The term “polydispersity” (or “dispersity” as recommended by IUPAC) is used to describe the degree of non-uniformity of a size distribution of particles. PDI is basically a representation of the distribution of size populations within a given sample. The numerical value of PDI ranges from 0.0 (for a perfectly uniform sample with respect to the particle size) to 1.0 (for a highly polydisperse sample with multiple particle size populations).

In some examples, the lipid particles can have a polydispersity index of 0.5 or less (e.g., 0.49 or less, 0.48 or less, 0.47 or less, 0.46 or less, 0.45 or less, 0.44 or less, 0.43 or less, 0.42 or less, 0.41 or less, 0.40 or less, 0.39 or less, 0.38 or less, 0.37 or less, 0.36 or less, 0.35 or less, 0.34 or less, 0.33 or less, 0.32 or less, 0.31 or less, 0.30 or less, 0.29 or less, 0.28 or less, 0.27 or less, 0.26 or less, 0.25 or less, 0.24 or less, 0.23 or less, 0.22 or less, 0.21 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, or 0.01 or less).

In some examples, the lipid particles can be substantially monodisperse. “Monodisperse” and “homogeneous size distribution,” as used herein, and generally describe a population of particles where all of the particles are the same or nearly the same size. As used herein, a monodisperse distribution refers to particle distributions in which 80% of the distribution (e.g., 85% of the distribution, 90% of the distribution, or 95% of the distribution) lies within 25% of the median particle size (e.g., within 20% of the median particle size, within 15% of the median particle size, within 10% of the median particle size, or within 5% of the median particle size).

Also disclosed herein is a composition comprising the lymphatic endothelial cell-specific lipid nanoparticles disclosed herein and a therapeutic agent. For example, in certain embodiments, the agent is a small molecule, organometallic compound, nucleic acid, protein, peptide, polynucleotide, metal, targeting agent, an isotopically labeled chemical compound, drug, vaccine, immunological agent, or an agent useful in bioprocessing. In some embodiments, the therapeutic agent is a polynucleotide. In certain embodiments, the polynucleotide is DNA or RNA. In some embodiments, the lymphatic endothelial cell-specific lipid nanoparticle of any preceding aspect comprises a nucleic acid (for example, DNA or RNA), a peptide, or a small molecule disposed therewithin. In some embodiments, the RNA is RNAi, dsRNA, siRNA, shRNA, miRNA, or antisense RNA.

The amount of nucleic acid in the nanoparticle can be from about 0.1%, about 0.2%, about 0.3%, about 0.4%, about 0.5%, about 0.6%, about 0.7%, about 0.8%, about 0.9%, about 1%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 15%, about 16%, about 17%, about 18%, about 19%, about 20%, about 22%, about 24%, about 26%, about 28%, about 30%, about 32%, about 34%, about 36%, about 38%, or about 40% of its nanoparticle weight. mRNA vaccines represent a promising alternative to conventional vaccine approaches because of their high potency, capacity for rapid development and potential for low-cost manufacture and safe administration through high yield in vitro transcription (reviewed in Pardi et al. mRNA Vaccines A New Era in Vaccinology, Nature Reviews Drug Discovery Volume 17, pages 261-279 (2018)). In some embodiments, the therapeutic agent comprises an mRNA-based vaccine. In some embodiments, the mRNA based vaccine is a conventional mRNA-based vaccine. In some embodiments, the mRNA-based vaccine encodes one or more antigen(s) of interest, e.g., a viral antigen(s). In some embodiments the mRNA-based vaccine comprises one or more of the following features: 5′ untranslated regions (UTR), 3′ UTR, polyA tail, one or more modified bases. In some embodiments, the mRNA based vaccine is a self-amplifying RNA, encoding one or more antigen(s) of interest. In some embodiments, the antigen of interest is of viral, bacterial, protozoan, fungal, or animal origin.

In some embodiments, the therapeutic agent includes a nucleic acid encoding an antigen designed to elicit a desired immune response. In some embodiments the nucleic acid encodes a vaccine antigen thus to elicit an immune response, and/or develop immune memory towards the encoded antigen. An antigen can include any protein or peptide that is foreign to the subject organism.

In some embodiments, the antigen is a viral antigen. A viral antigen can be isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (EBOV) (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenza virus A, such as H1N1 strain, and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3. Viral antigens may be derived from a particular strain such as a papilloma virus, a herpes virus, i.e. herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

In some embodiments, the antigen is a bacterial antigen. Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

In some embodiments, the antigen is a parasite antigen. Exemplary parasite allergens, include but are not limited to, Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydialpsittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein. In some embodiments, the parasite antigen is one or more antigens from a protozoan, such as one or more protozoans from the genus Toxoplasma, for example T. gondii and species from a related genus, such as Neospora, Hammondia, Frenkelia, Isospora and Sarcocystis. Examples of antigens derived from T. gondii include the GRA6, ROP2A, ROP18, SAG1, SAG2A and AMA1 gene products.

In some embodiments, the antigen is a cancer antigen. Cancer antigens can be antigens expressed only on tumor cells and/or required for tumor cell survival. In some embodiments, the antigen is a tumor antigen. There are many classes of tumor antigens, including, but not limited to, oncogene expression products, alternatively spliced expression products, mutated gene products, over-expressed gene products, aberrantly expressed gene products, antigens produced by an oncogenic viruses, oncofetal antigens, as well as proteins with altered cell surface glycolipids, and proteins having altered glycosylation profiles. Examples of tumor antigens include tumor-associated or tumor-specific antigens, such as, but not limited to, alpha-actinin-4, Alphafetoprotein (AFP), Bcr-Abl fusion protein, Carcinoembryonic antigen (CEA), CA-125, Casp-8, beta-catenin, cdc27, cdk4, cdkn2a, coa-1, dek-can fusion protein, epithelial tumor antigen, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARa fusion protein, PTPRK, K-ras, N-ras, Triosephosphate isomeras, Bage-1, Gage 3, 4, 5, 6, 7, GnTV, Herv-K-mel, Lage-1, Melanoma-associated antigen (MAGE); Mage-A1, 2, 3, 4, 6, 10, 12, Mage-C2, NA-88, NY-Eso-1/Lage-2, SP 17, SSX-2, and TRP2-Int2, MelanA (MART-I), gp100 (Pmel 17), tyrosinase, TRP-1, TRP-2, MAGE-1, MAGE-3, BAGE, GAGE-i, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, b-Catenin, CDK4, Mum-1, p16, TAGE, PSMA, PSCA, CT7, telomerase, 43-9F, 5T4, 791Tgp72, a-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, MUC-1, NB\70K, NY-CO-1, RCAS1, SDCCAG16, TA-90 (Mac-2 binding proteincyclophilin C-associated protein), TAAL6, TAG72, TLP, tyrosinase, and TPS.

Also disclosed herein is a method of delivering an agent to a lymphatic system in a subject, comprising administering to the subject an effective amount of a composition comprising the lymphatic endothelial cell-specific lipid nanoparticle disclosed herein and a therapeutic agent disposed therewithin. In some aspects, the nanoparticles comprises a liposome, a multilamellar vesicle, or a solid lipid nanoparticle.

Also disclosed herein is a method of treating a lymphatics-associated disorder in a subject, comprising administering to the subject a therapeutically effective amount of the LEC-specific lipid nanoparticle disclosed herein. In some examples, the lymphatics-associated disorder is a cardiovascular disease, lymphatic vascular injury, auto-immune disease, or brain injury. In some embodiments, the lymphatic endothelial cell-specific lipid nanoparticle comprises a VEGF-C polynucleotide.

“VEGF-C” refers herein to a polypeptide that, in humans, is encoded by the VEGFC gene. In some embodiments, the VEGF-C polypeptide is that identified in one or more publicly available databases as follows: HGNC: 12682, NCBI Entrez Gene: 7424, Ensembl: ENSG00000150630, OMIM®: 601528, UniProtKB/Swiss-Prot: P49767.

It should be understood and herein contemplated that the lymphatic endothelial cell-specific lipid nanoparticle (e.g., a nanoparticle comprising a VEGF-C polynucleotide) disclosed herein does not promote cancer metastasis.

Also disclosed herein is a method of enhancing lymphangiogenesis in a subject, comprising administering to the subject a therapeutically effective amount of the lymphatic endothelial cell-specific lipid nanoparticle disclosed herein.

Also disclosed herein is an mRNA vaccine comprising the lymphatic endothelial cell-specific lipid nanoparticle disclosed herein and an mRNA.

Also disclosed herein is a method of preventing or treating a disorder or an infection in a subject, comprising administering to the subject a therapeutically effective amount of the mRNA vaccine disclosed herein.

In some embodiments, the vaccine disclosed herein can be used for preventing or treating a viral infection, a bacterial infection, a parasitic infection, or a yeast infection.

In some embodiments, the vaccine disclosed herein can be used for preventing or treating a cancer. A cancer can be selected from, but is not limited to, a hematologic cancer, lymphoma, colorectal cancer, colon cancer, lung cancer, a head and neck cancer, ovarian cancer, prostate cancer, testicular cancer, renal cancer, skin cancer, cervical cancer, pancreatic cancer, and breast cancer. In one aspect, the cancer comprises a solid tumor. In another aspect, the cancer is selected from acute myeloid leukemia, myelodysplastic syndrome, chronic myeloid leukemia, acute lymphoblastic leukemia, myelofibrosis, multiple myeloma. In another aspect, the cancer is selected from a leukemia, a lymphoma, a sarcoma, a carcinoma and may originate in the marrow, brain, lung, breast, pancreas, liver, head and neck, skin, reproductive tract, prostate, colon, liver, kidney, intraperitoneum, bone, joint, and eye.

Also disclosed herein is a pharmaceutical composition comprising the LEC-specific lipid nanoparticle disclosed herein and a pharmaceutically acceptable carrier.

### Examples

The following examples are set forth below to illustrate the compositions, methods, and results according to the disclosed subject matter. The examples are not intended to be inclusive of all aspects of the subject matter disclosed herein, but rather to illustrate representative methods and results. These examples are not intended to exclude equivalents and variations of the present invention which are apparent to one skilled in the art.

### Example 1

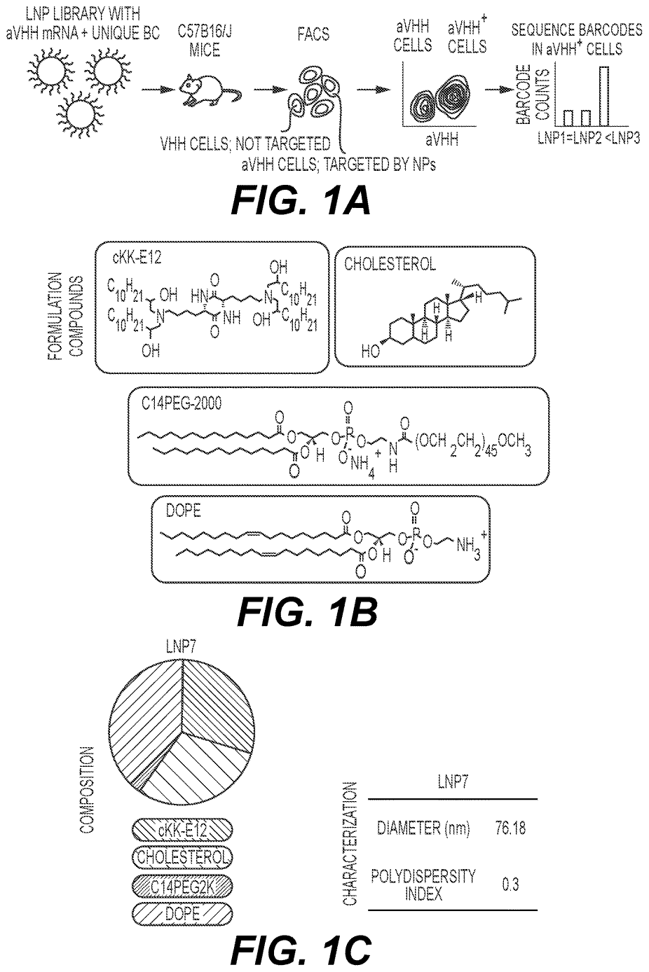

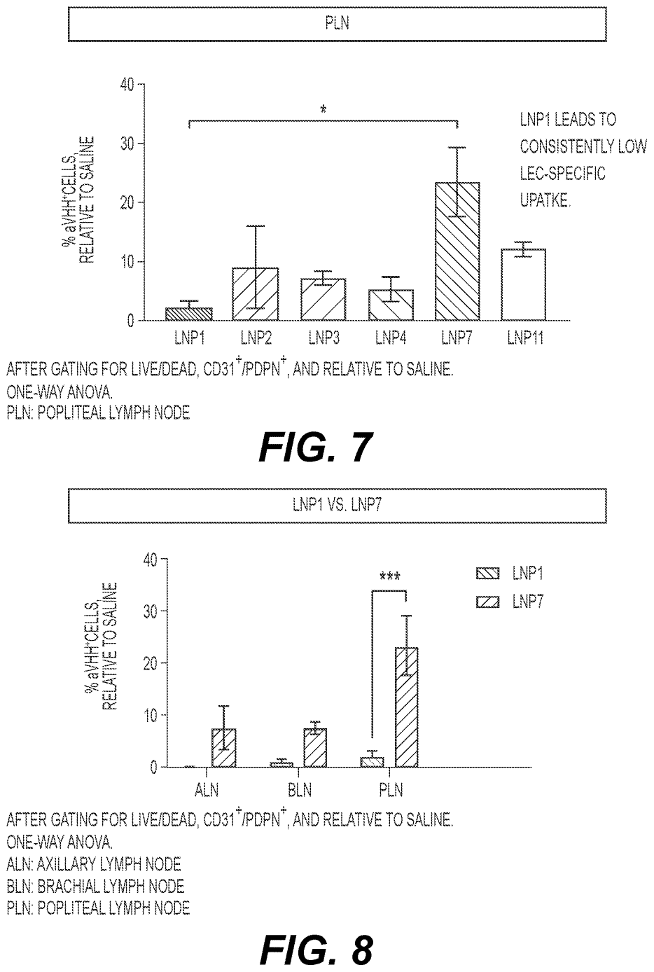

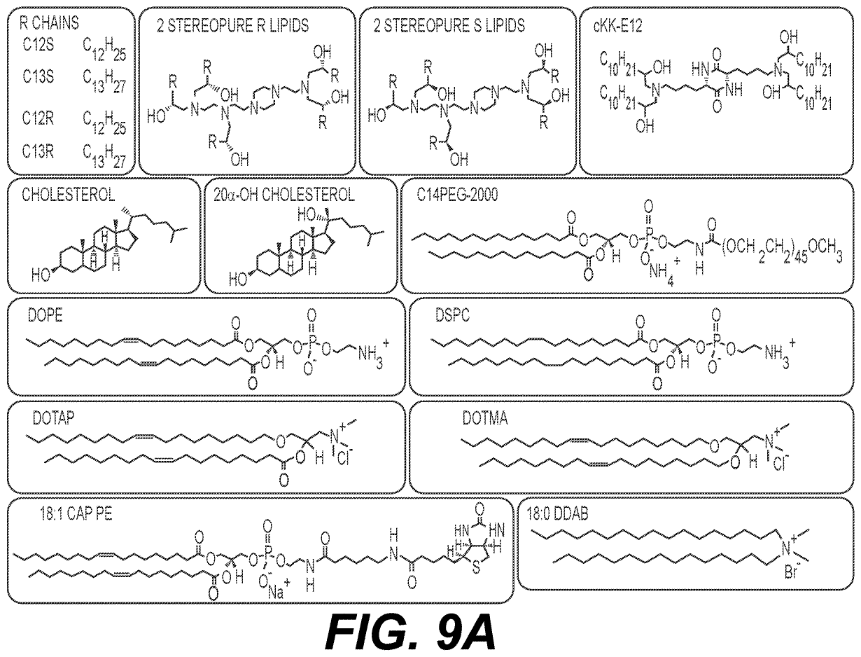

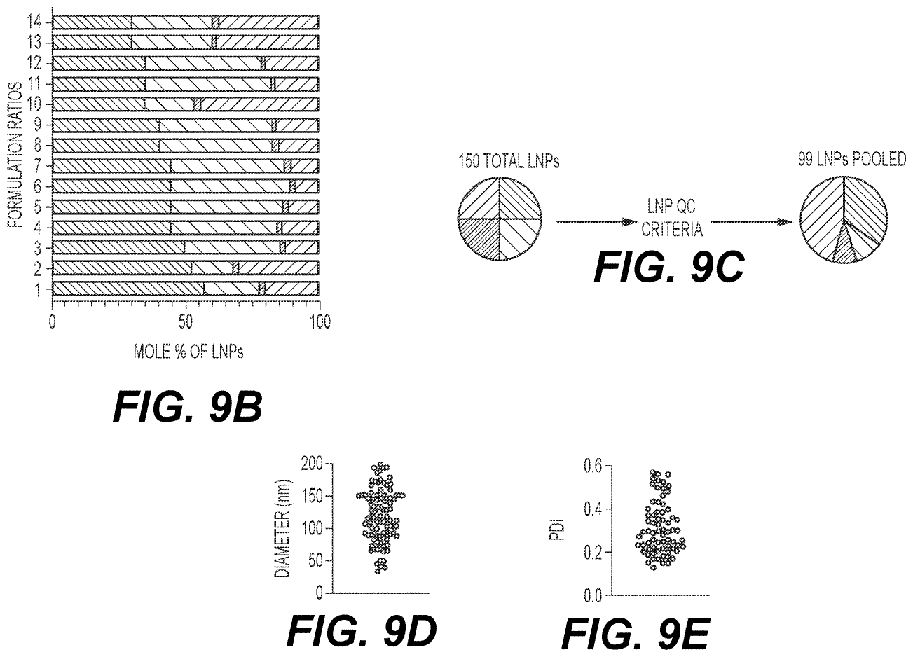

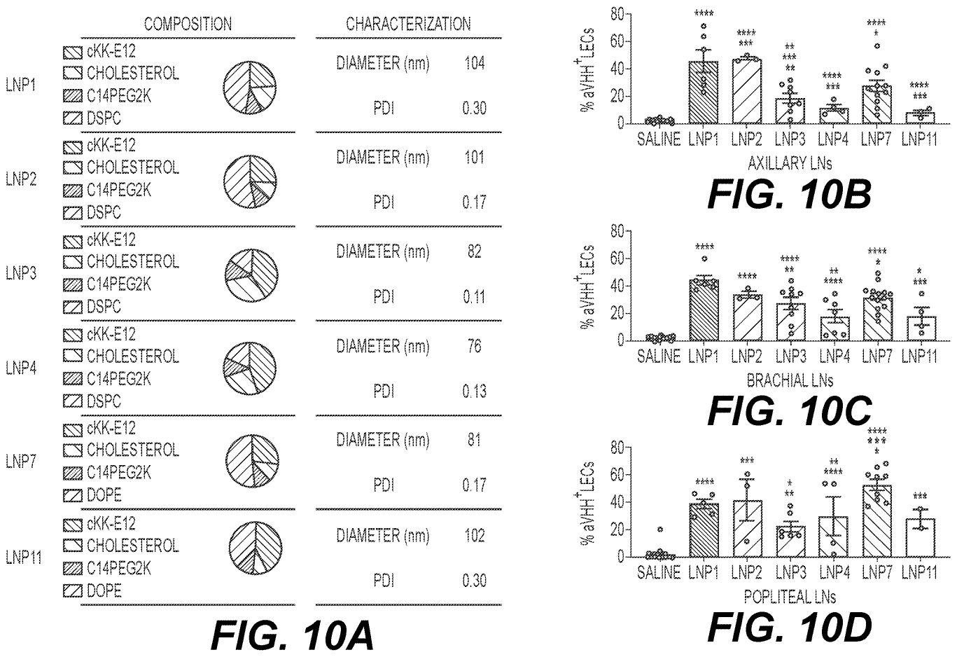

LNP candidates were initially screened by injecting different formulations into mouse limbs to identify LNPs that target lymphatic endothelial cells (LECs) with high specificity and can be promising candidates as a therapeutic vehicle for functional mRNA delivery of vascular endothelial growth factor C (VEGF-C). Utilizing FIND (Fast Identification of Nanoparticle Delivery), which is a method capable of simultaneously quantifying how >100 LNPs deliver mRNA that is translated into functional protein to various cell types in vivo, this experiment first screened a library of 80 LNPs with varying compositions of lipid-amine compounds, alkyl-tailed PEG, cholesterol, and helper lipids (i.e., DOPE, DSPC, DOTAP). For LNP screenings, each chemically distinct LNP was formulated to carry an mRNA reporter for a protein not normally synthesized by LECs (aVHH) and a unique DNA barcode (FIG. 1A). LNPs were intradermally injected into mouse paws and footpads. After 12-16 hours, the downstream lymph nodes (LNs) (i.e., axillary LN (ALN), brachial LN (BLN), and popliteal LN (PLN)) were collected, digested, and run through flow cytometry. Sufficient numbers of viable cells after sorting (as determined by Live/Dead gating) were available from each corresponding isolation. To isolate LECs, the experiment was sorted for CD31+/PDPN+ cells, where CD31 is a well-established EC marker and PDPN is a LEC marker. LECs that took up LNPs that delivered functional genetic cargo were identified through the expression of aVHH (aVHH+). Several hundreds of LECs that took up LNPs were sorted for sequencing. This experiment identified the specific DNA barcodes of the LNPs within the sorted LECs and thus the corresponding LNPs formulations that lead to successful lymphatic-specific targeting. The percentage of aVHH+ cells relative to a saline control is shown in FIGS. 5-7. FIG. 8 shows a comparison of LNP1 (left) and LNP7 (right) for each of the LN cell types.

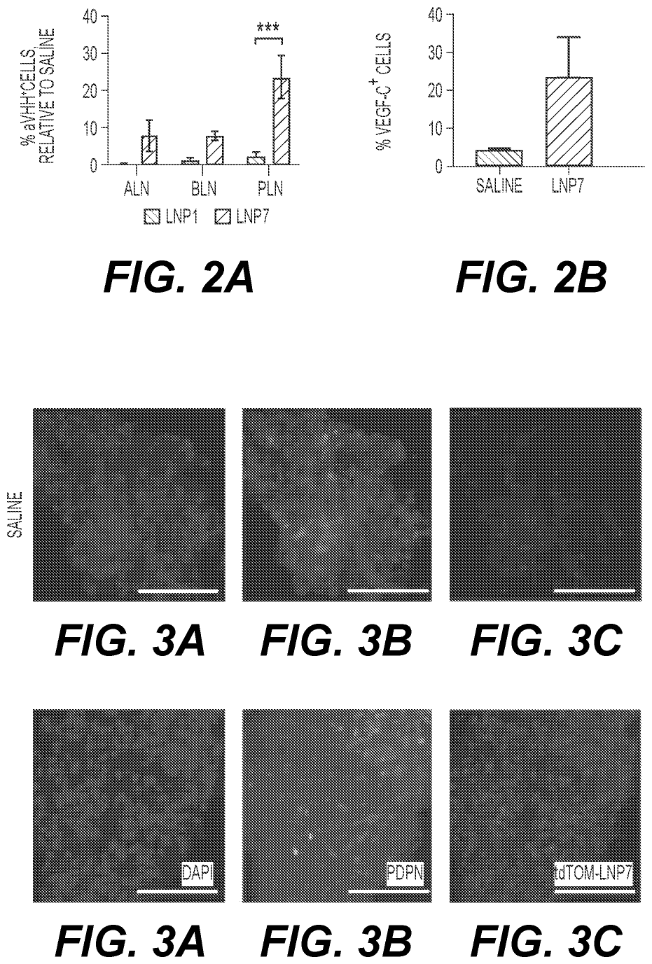

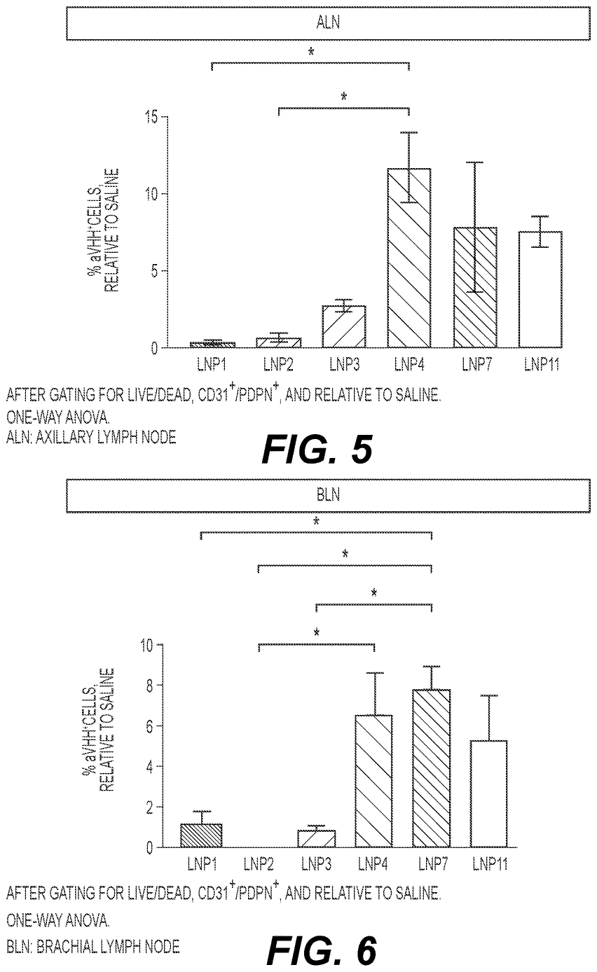

These screenings identified LEC-specific LNP candidates that lead to the highest and lowest LEC-specific uptake. The lymphatic targeting of one of the more promising candidates (LNP7) along with a poorly targeting candidate (LNP1) was tested. LNP7 contains the following compounds: 100% Ethanol (37.04% v/v), lipomer cKK-E12 ([10]; 17.04% v/v), Cholesterol ([5]; 6.84% v/v), lipid-anchored poly-(ethylene glycol) C14PEG2K ([5]; 6.6% v/v), and phospholipid dioleoylphosphatidylethanolamine (DOPE) ([5]; 32.48% v/v) (FIGS. 1B-1D). Thus, this study identified a stable LNP (LNP7; Average Diameter=76.18 nm, Average Polydispersity Index=0.3) that targets LECs with high specificity. Specifically, the flow cytometry data has identified LNP7 as the lead LEC-specific candidate with the highest uptake of aVHH compared to LNP1 which leads to low LEC-specific uptake, i.e., percentage of LEC-specific LNP uptake in PLNs: 23.5% of LNP7 and 2.2% of LNP1 (p=0.0009) (FIG. 2A). This LNP (i.e., LNP7) serves as a promising candidate for functional mRNA delivery in LECs, which has never been done before. LNP7 was then loaded with a synthetic VEGF-C mRNA (unprocessed form) and delivered to the paws and footpads of mice to demonstrate the LNP can increase VEGF-C synthesis in LEC. Flow cytometry data showed that administration of LNP7 loaded with VEGF-C mRNA led to a higher number of VEGF-C+ cells (23.6%) compared to administration of saline (4.2%) demonstrating the successful delivery of VEGF-C mRNA into LECs by LNP7 (FIG. 2B). Compositions of several studied LNPs as well as characteristics thereof are shown in FIGS. 4A-4B.

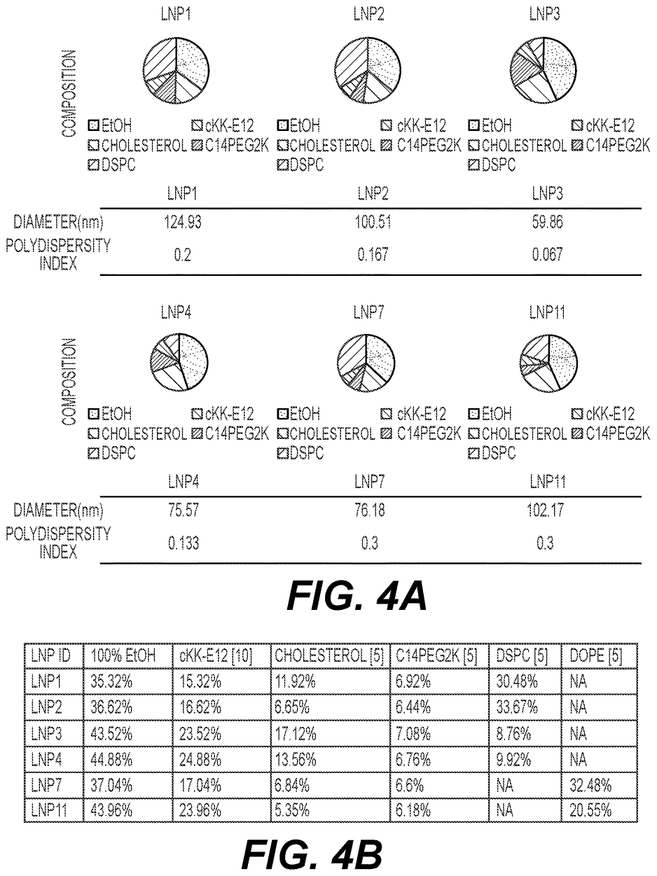

Additional experiments were performed where a fluorescent reporter mRNA was delivered to a tdTom (Cre) mouse, that drives a promoter when the mRNA cargo is successfully delivered to the cells. This experiment dissected out the afferent lymphatic vessel that drains to the PLN and then performed whole mount imaging to demonstrate the uptake and delivery of cargo to the LECs that are lining the collecting lymphatic vessel (FIG. 3A-3F).

In summary, this study has developed and validated several candidate LNP formulations with high lymphatic specificity when administered intradermally, capable of delivering functional VEGF-C mRNA to lymphatics.

This study demonstrates for the first time on lymphatics an innovative new platform for targeting and functional delivery of mRNA to lymphatics through loco-regional dermal or subcutaneous delivery. Lipid-encapsulated drug formulations provide advantages over traditional drug-delivery methods. For example, some lipid-based formulations provide longer half-lives in vivo, superior tissue targeting, and decreased toxicity. Notably, mRNA-based platforms offer a transient and less immunogenic method of delivery compared to protein therapeutics or adenovirus and adeno-associated virus-based platforms, thus suggesting a promising therapeutic avenue.

The uses of this platform include but are not limited to the following applications:

In the context of regenerating the lymphatic vasculature post-injury, VEGF-C has been a promising growth factor that enhances lymphangiogenesis. Notably, VEGF-C mRNA delivery in LECs using LNPs can lead to the transient enhancement of VEGF-C, thus alleviating problems associated with the long-term upregulation of VEGF-C observed with other therapeutics—permanent upregulation of VEGF-C has been shown to promote cancer cell metastasis. Targeting LECs that take up the LNP to be the source of VEGF-C can increase the network capacity by enhancing the growth of pre-existing functional vessels.

This LNP platform can serve as an alternative to the mRNA vaccine platforms. When ribosomes read the vaccine mRNA, pieces of the viral surface protein are made. These pieces are then displayed on the surface of the dendritic cell. The dendritic cell travels to a nearby lymph node (LN), where it presents the surface proteins to other cells of the immune system.

Notably, there is a higher number of LECs that reside within the LNs compared to dendritic cells and LECs possess the capability to present antigens to T-cells. Alternatively, lymphatic endothelial cells in the lymph node have also been shown to play an important role in the presentation of self-antigen for the maintenance of tissue tolerance. Thus, the delivery of functional mRNA in LECs via the use of LNP7 indicates a promising and novel avenue for therapeutic purposes in which one seeks to utilize LEC to promote either tissue tolerance or adaptive immunity against foreign antigens.

### Example 2

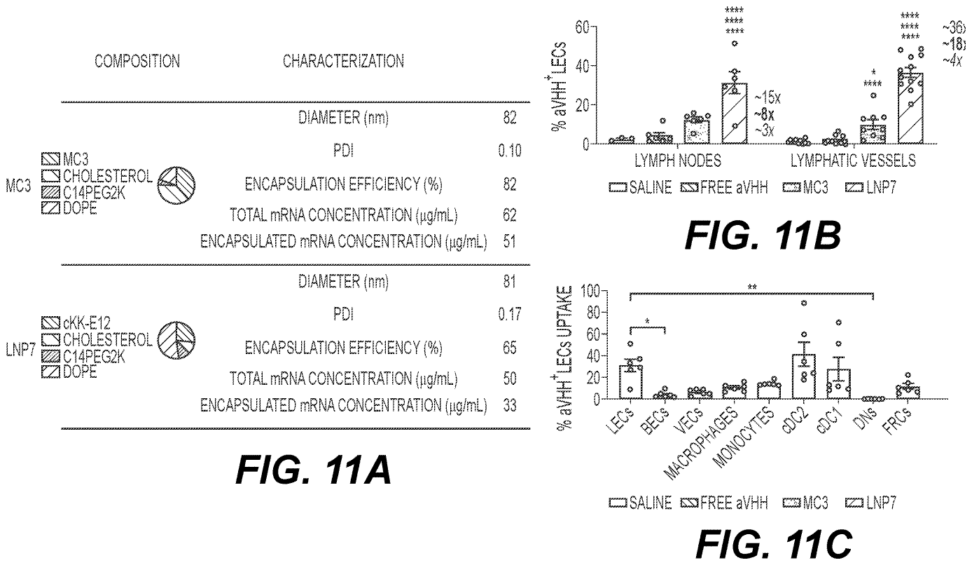

A panel of LNP candidates were prepared similar to Example 1. Each of the compounds was formulated using 14 molar ratios as shown in FIGS. 9A-9B. After formulation of each of the LNP recipes, the corresponding citrate and lipid phases were combined in a microfluidic device. The quality of each of the LNPs was evaluated using dynamic light scattering (DLS) to measure the diameter and polydispersity index (PDI), nanodrop to measure the mRNA concentration, and encapsulation assay to quantify the corresponding encapsulation efficiency.

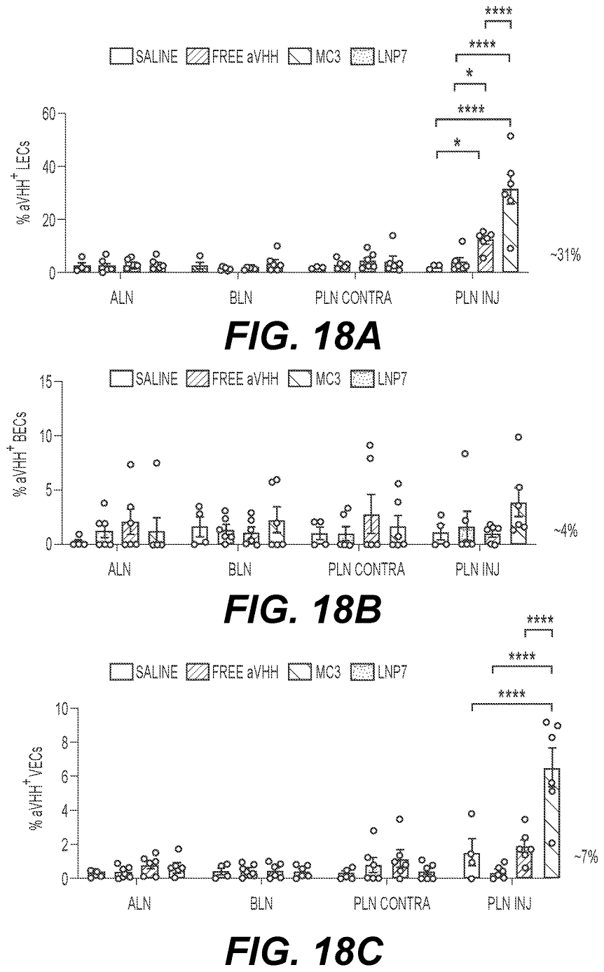

As illustrated in FIG. 15, LNP7 displayed a comparable hydrodynamic diameter and Polydispersity indexes (PDI) to the MC3 control with the respective cargo. After evaluation, 99 of the 150 formulated LNPs passed the quality control (QC) criteria, with a diameter less than 200 nm as well as a stable autocorrelation curve. FIG. 9C. The hydrodynamic diameters and PDI of all administered LNPs; the diameter of the LNP pooled control is within the range of the LNPs composing the pool. (FIGS. 9D-9E).

Several lead candidates for LEC-specific LNPs were then identified and characterized based on the percentage of aVHH+ LECs after gating for Live/Dead, CD31⁺/PDPN⁺. LNP libraries with aVHH mRNA and unique DNA barcode were administered to C57Bl6/J mice.

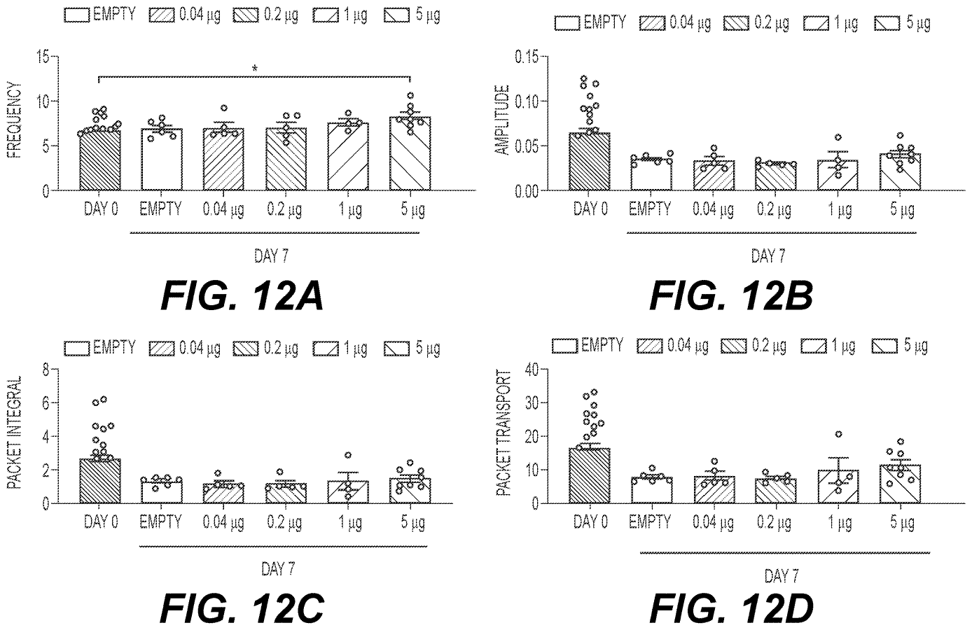

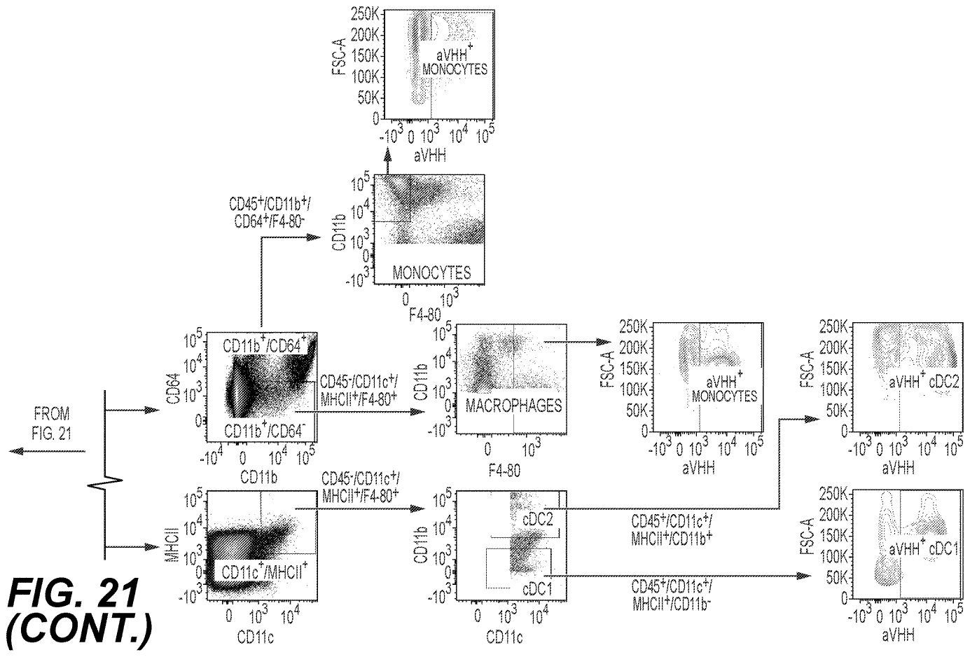

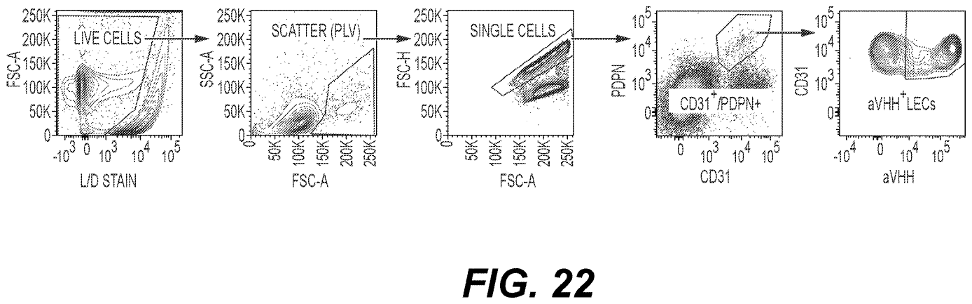

FIG. 10A. aVHH+ cells were isolated by FACS and DNA barcodes are sequenced. A representative gating strategy for FACS for various cell populations present in LNs is shown in FIGS. 21-22 and includes: LECs: CD45⁻/CD31⁺/PDPN⁺, VECs: CD45⁻/CD31⁺/PDPN⁻/CD54⁺, BECs: CD45⁻/CD31⁺/PDPN⁻/CD309⁺, FRCs: CD45⁻/CD31⁻/PDPN⁺, DNs: CD45⁻/CD31⁻/PDPN, Monocytes: CD45⁺/CD11b⁺/CD64⁺/F4-80⁻, Macrophages: CD45⁺/CD11b⁺/CD64⁻/F4-80⁺, cDC1: CD45⁺/CD11c⁺/MHCII⁺/CD11b⁺, and cDC2: CD45⁺/CD11c⁺/MHCII⁺/CD11b⁺.

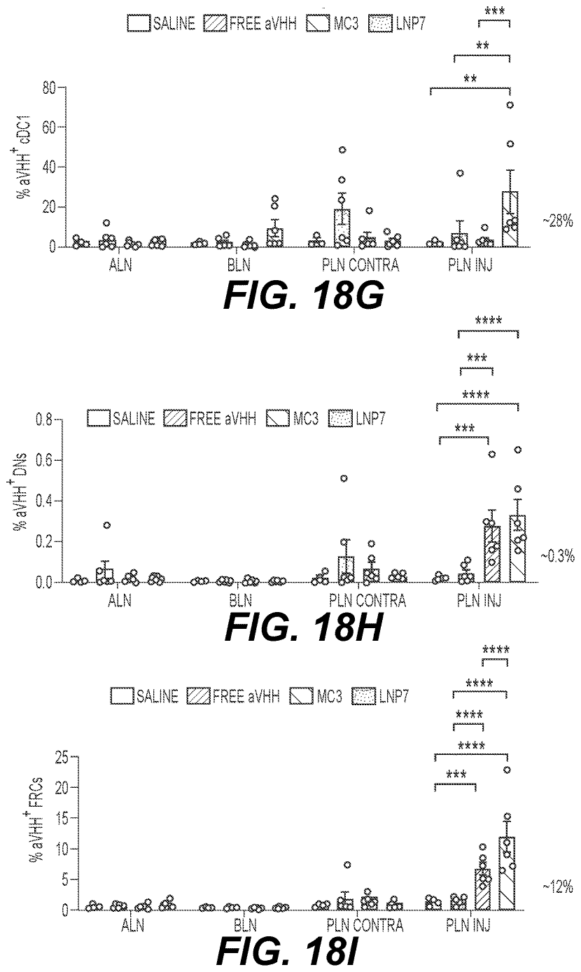

Intradermal (ID) injection was shown to result in a higher percentage of aVHH+ cells. (FIG. 17). Thus, LNPs were ID injected in C57Bl/6J mice and 12-16 hrs later the downstream LNs or LVs were collected for flow cytometry. Control animals were ID injected with appropriate volumes of saline. FIGS. 10B-10D classify the proportion of LECs axillary LNs (ALN), brachial LNs (BLN), and popliteal LNs (PLN) that had successfully taken up saline, LNP1, LNP2, LNP3, LNP4, LNP7, and LNP11. The aVHHI+ cell counts corresponding to each LN type for the lead candidates is shown in Table 1.

TABLE 1
Average LEC count and LEC/aVHH⁺ cell count for lead LEC-specific
LNP candidates - LNP1, LNP2, LNP3, LNP4, LNP7, and LNP11.

| | | | aVHH⁺ Cell |
|---|---|---|---|
| Type of LN | Administration | LEC Count | Count |
| ALN | Saline | 2182 | 37 |
| | LNP1 | 1022 | 543 |
| | LNP2 | 1467 | 697 |
| | LNP3 | 948 | 134 |
| | LNP4 | 997 | 134 |
| | LNP7 | 2757 | 977 |
| | LNP11 | 372 | 37 |
| BLN | Saline | 2016 | 39 |
| | LNP1 | 2139 | 376 |
| | LNP2 | 2169 | 715 |
| | LNP3 | 1971 | 572 |
| | LNP4 | 3319 | 159 |
| | LNP7 | 2204 | 743 |
| | LNP11 | 3631 | 239 |
| PLN | Saline | 95 | 2 |
| | LNP1 | 388 | 139 |
| | LNP2 | 105 | 31 |
| | LNP3 | 771 | 144 |
| | LNP4 | 118 | 49 |
| | LNP7 | 782 | 292 |
| | LNP11 | 230 | 52 |

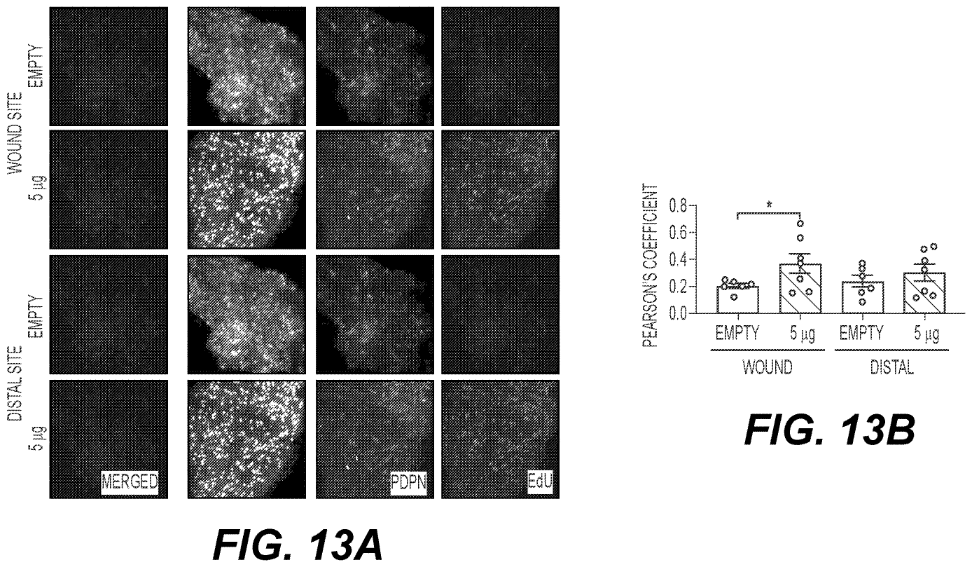

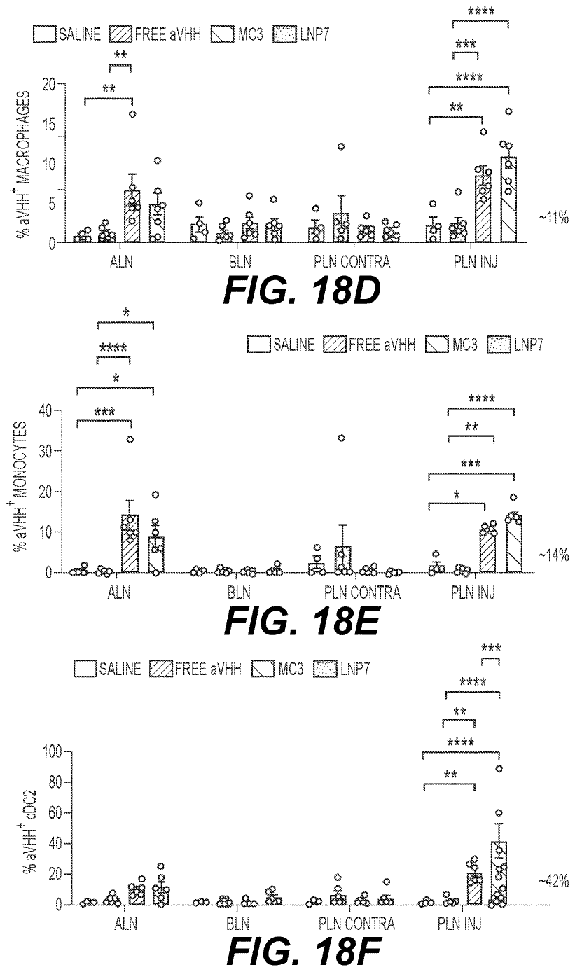

Next, several lead LNP compositions were tested against MC3-based lipid nanoparticles with aVHH and VEGF-C mRNA cargo to characterize the feasibility of including multiple different mRNA components. The formulation compounds, composition, hydrodynamic diameter (nm), PDJ, encapsulation efficiency (0%), total mRNA concentration (μg/mL), encapsulated mRNA concentration (μg/mL) between MC3 and LNP7 are shown in FIG. 11A. Graphs showing the percentage of aVHH⁺ LECs (after gating for Live/Dead, CD45⁻/CD31⁺/PDPN⁺) from popliteal LNs and LVs that have successfully taken up saline, free aVHH, MC3 (standard positive control LNP) and LNP7 and the percentage of aVHH⁺ uptake of LNP7 by different cell types, namely LECs, BECs, VECs, Macrophages, Monocytes, cDC2, cDC1, DNs, and FRCs in PLN Inj. are included in FIGS. 11B-11C. As shown in FIG. 16, the hydrodynamic diameter (nm), PDI, encapsulation efficiency (%), total mRNA concentration (μg/mL), encapsulated mRNA concentration (μg/mL) was comparable between MC3 and LNP7 LNPs having different mRNA cargos, i.e., aVHH mRNA and VEGFC mRNA. Notably LNP7 saw encapsulation efficiencies of 65% and 43% with an encapsulated mRNA concentration of 33 g/mL and 17 g/mL for aVHH and VEGF-C, respectively.

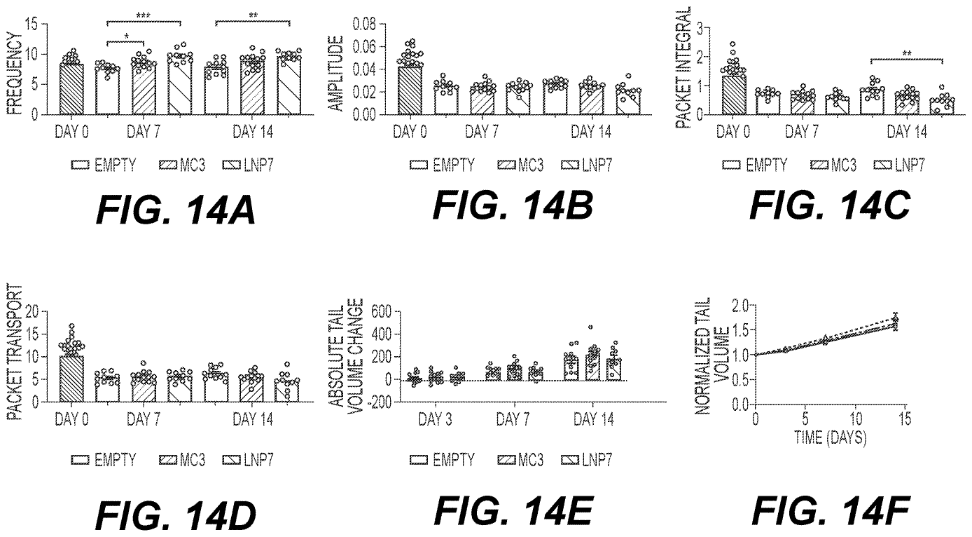

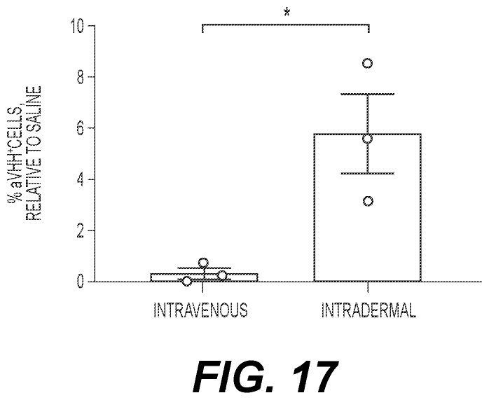

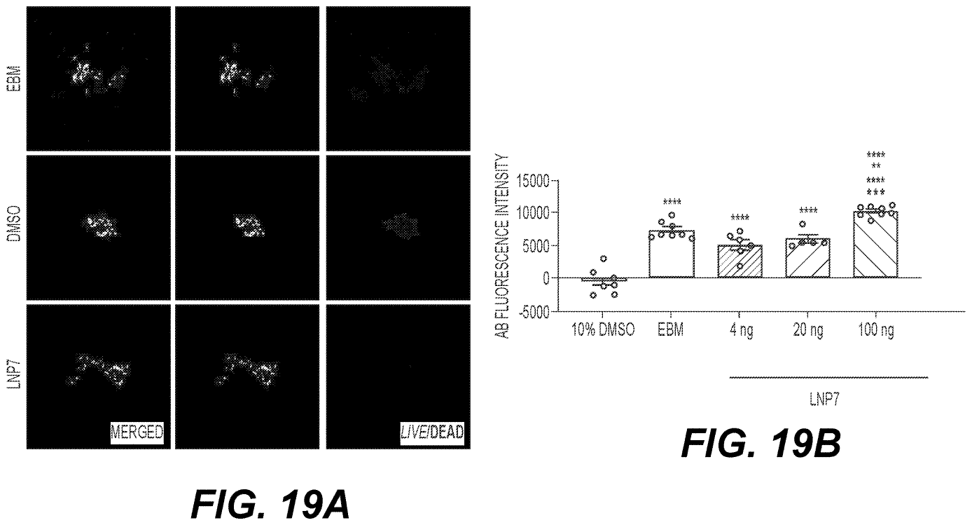

Following similar procedures to those discussed above, MC3 and LNP7 LNPs were then administered to determine their uptake by various cell populations residing in LNs. Illustrated by FIGS. 18A-18I, LNP7 demonstrated high specificity and uptake relative to MC3, free aVHH, and saline in various LEC populations. The amount of cell counts and aVHH⁺ LECs (after gating for Live/Dead, CD45⁻/CD31⁺/PDPN⁺) from popliteal LNs that have successfully taken up saline, free aVHH, MC3, and LNP7 is reproduced in Tables 2-3. VEGFC mRNA significantly increased the frequency 14-days after mouse tail lymphedema surgery in vivo. FIGS. 12A-12D and FIGS. 14A-14F.

TABLE 2
Average cell count for the various cell types found in ALN, BLN, PLN-contralateral,
PLN-injected, and PLV for saline-, free aVHH, MC3-, and LNP7-injected animals.

| | | Cell Type Count | | | | | | | | |
|---|---|---|---|---|---|---|---|---|---|---|
| Type of LN | Administration | Macrophages | Monocytes | cDC2 | cDC1 | DNs | FRCs | LECs | BECs | VECs |
| ALN | Saline | 4036 | 1294 | 2914 | 1008 | 114907 | 6175 | 7099 | 237 | 1269 |
| | Free aVHH | 4970 | 1606 | 4155 | 1356 | 270150 | 6045 | 10462 | 744 | 3796 |
| | MC3 | 4013 | 67256 | 4264 | 2682 | 155750 | 7848 | 7993 | 145 | 1995 |
| | LNP7 | 4665 | 10492 | 6018 | 3885 | 337655 | 8294 | 7802 | 167 | 1879 |
| BLN | Saline | 7113 | 2666 | 8368 | 2474 | 105104 | 13394 | 10007 | 296 | 2267 |
| | Free aVHH | 10295 | 4097 | 11666 | 3698 | 168662 | 15316 | 16065 | 574 | 6351 |
| | MC3 | 8562 | 6471 | 12017 | 4630 | 224232 | 21738 | 15810 | 249 | 5418 |
| | LNP7 | 7940 | 4129 | 12104 | 3390 | 227405 | 14637 | 10541 | 310 | 3433 |
| PLN | Saline | 1790 | 728 | 1417 | 541 | 18325 | 2056 | 3034 | 3475 | 488 |
| Contralateral | Free aVHH | 1069 | 439 | 915 | 636 | 15212 | 1273 | 3686 | 45 | 624 |
| | MC3 | 1302 | 651 | 1486 | 719 | 13422 | 2742 | 1267 | 28 | 381 |
| | LNP7 | 1784 | 927 | 1934 | 912 | 29328 | 2412 | 4061 | 62 | 1111 |
| PLN Injected | Saline | 2054 | 599 | 1330.25 | 336 | 28092 | 1791 | 3272 | 56.25 | 632 |
| | Free aVHH | 4425 | 1737 | 1680 | 874 | 27735 | 1990 | 4153 | 70.5 | 1002 |
| | MC3 | 7739 | 25134 | 2737 | 2445 | 34778 | 4618 | 4220 | 72 | 1091 |
| | LNP7 | 7568 | 25225 | 3605 | 2586 | 69388 | 5089 | 5042 | 85 | 1058 |
| PLV | Saline | NA | NA | NA | NA | NA | NA | 185 | NA | NA |
| | Free aVHH | NA | NA | NA | NA | NA | NA | 188 | NA | NA |
| | MC3 | NA | NA | NA | NA | NA | NA | 189 | NA | NA |
| | LNP7 | NA | NA | NA | NA | NA | NA | 269 | NA | NA |

TABLE 3
Average aVHH⁺ cell count for the various cell types found in ALN, BLN, PLN-contralateral,
PLN-injected, and PLV for saline-, free aVHH, MC3-, and LNP7-injected animals.

| | | aVHH⁺ Cell Count | | | | | | | | |
|---|---|---|---|---|---|---|---|---|---|---|
| Type of LN | Administration | Macrophages | Monocytes | cDC2 | cDC1 | DNs | FRCs | LECs | BECs | VECs |
| ALN | Saline | 43 | 3 | 31 | 21 | 8.5 | 35 | 89 | 1 | 3 |
| | Free aVHH | 45 | 3 | 106 | 37 | 52 | 38 | 406 | 3 | 15 |
| | MC3 | 320 | 876 | 437 | 31 | 31 | 44 | 263 | 1 | 18 |
| | LNP7 | 201 | 1142 | 781 | 82 | 55 | 62 | 192 | 1 | 14 |
| BLN | Saline | 160 | 5 | 492 | 246 | 6 | 49 | 156 | 5 | 13 |
| | Free aVHH | 150 | 10 | 170 | 75 | 10 | 67 | 226 | 115 | 44 |
| | MC3 | 287 | 20 | 162 | 47 | 14 | 65 | 254 | 3 | 41 |
| | LNP7 | 152 | 28 | 578 | 441 | 14 | 57 | 409 | 7 | 21 |
| PLN | Saline | 50 | 2.5 | 37 | 52 | 3 | 17 | 65 | 0.5 | 2 |
| Contralateral | Free aVHH | 37 | 2 | 97 | 149 | 5 | 9 | 158 | 1 | 12 |
| | MC3 | 25 | 4 | 43 | 35 | 12 | 68 | 45 | 1 | 4 |
| | LNP7 | 28 | 2 | 82 | 75 | 8 | 16 | 89 | 2 | 8 |
| PLN Injected | Saline | 73 | 2 | 31 | 13 | 6 | 21 | 98 | 1 | 7 |
| | Free aVHH | 78 | 11 | 29 | 11 | 10 | 21 | 79 | 1 | 8 |
| | MC3 | 772 | 2755 | 521 | 86 | 78 | 296 | 591 | 1 | 20 |
| | LNP7 | 888 | 3681 | 1677 | 978 | 202 | 568 | 1690 | 3 | 77 |
| PLV | Saline | NA | NA | NA | NA | NA | NA | 3 | NA | NA |
| | Free aVHH | NA | NA | NA | NA | NA | NA | 3 | NA | NA |
| | MC3 | NA | NA | NA | NA | NA | NA | 18 | NA | NA |
| | LNP7 | NA | NA | NA | NA | NA | NA | 104 | NA | NA |

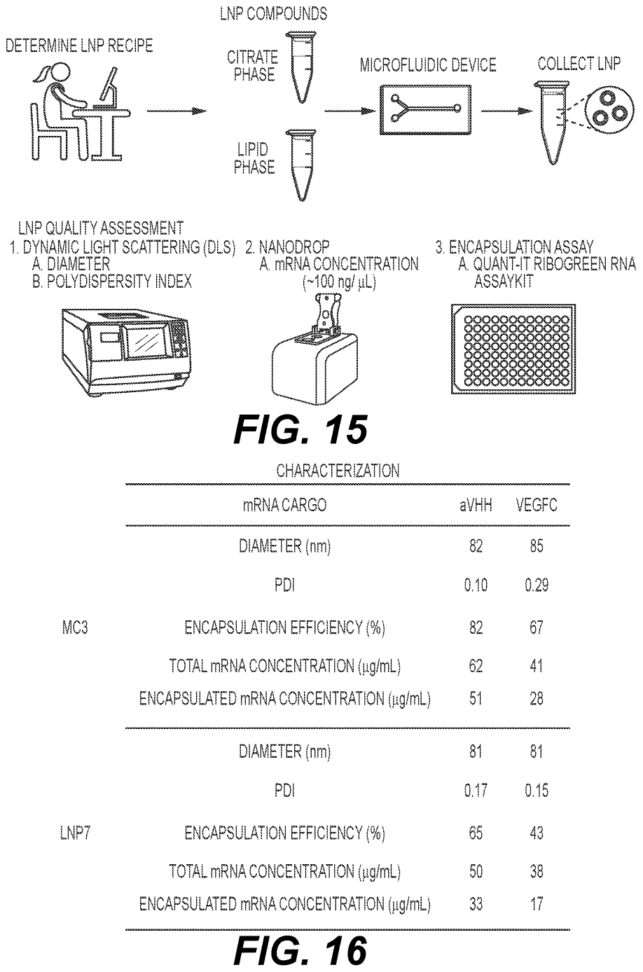

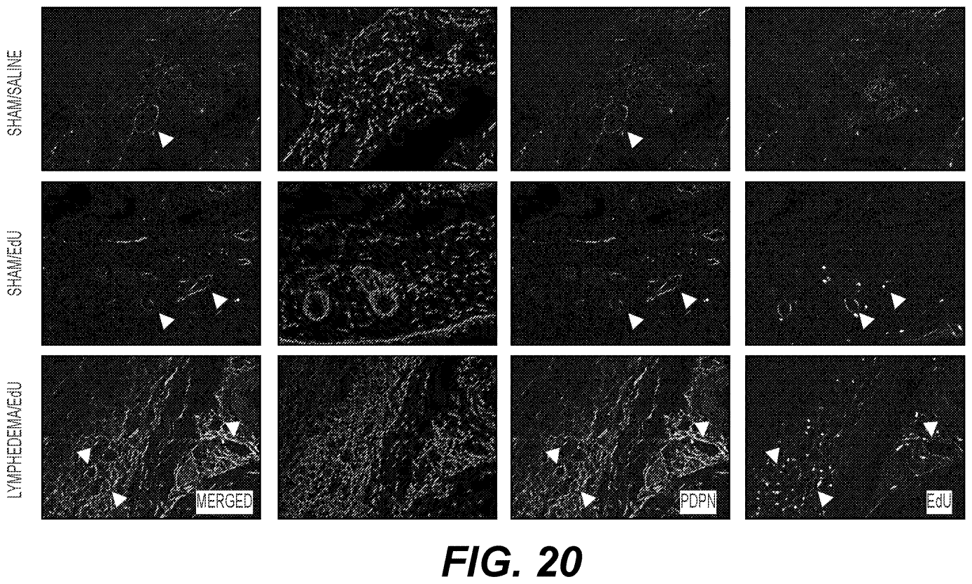

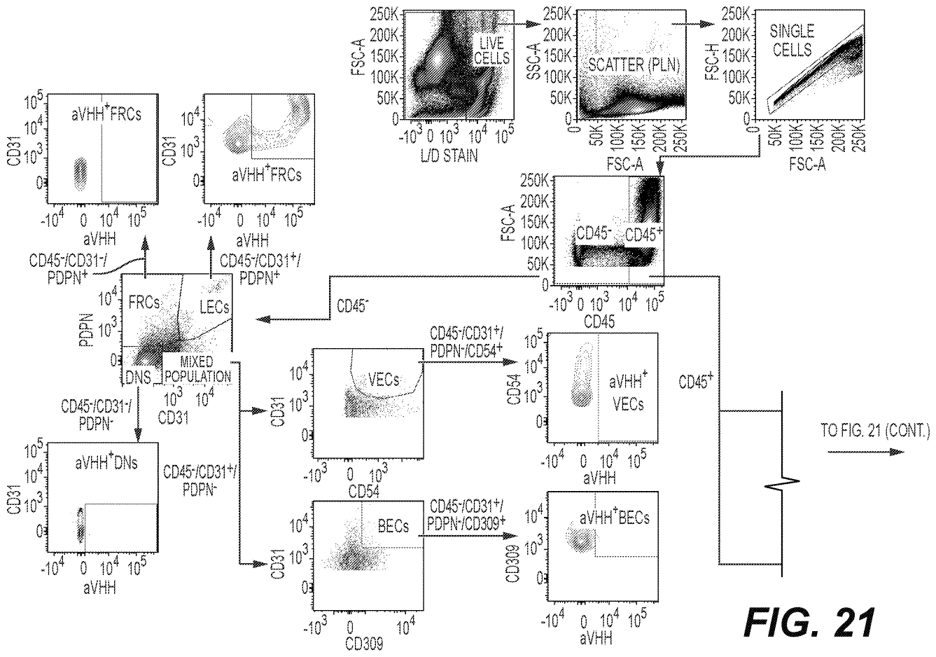

VEGFC mRNA overexpression was shown to significantly increase the PDPN/EdU colocalization in the wound site after mouse tail lymphedema surgery in vivo. Representative immunofluorescence micrographs were obtained of merged, DAPI, PDPN, and EdU in tail sections for empty and 5 g in wound and distal sites 7 days post-surgery. (FIG. 13A). FIG. 13B shows a comparison of the Pearson's coefficient for empty and 5 μg 7-days post-surgery in the wound and distal sites. FIG. 20 shows IHC controls in tail LVs. Immunofluorescence micrographs of tail LV segments were collected for sham mice injected with saline (negative control), sham mice injected with EdU, and lymphedema mice injected with EdU 7 days post-surgery for merged, DAPI, PDPN, and EdU. LEC-specific LNP (i.e., LNP7) was further demonstrated to be non-toxic in the studied cell population. Immunofluorescence micrographs were obtained of human LECs in EBM-, DMSO-, and LNP7-treated cells for merged, DAPI, and Live/Dead as shown in FIG. 19A. Quantification of the L/D staining and alamarBlue fluorescence intensity (FIG. 19B) indicated that LNP7 displayed similar fluorescence to the control.

### Example 3

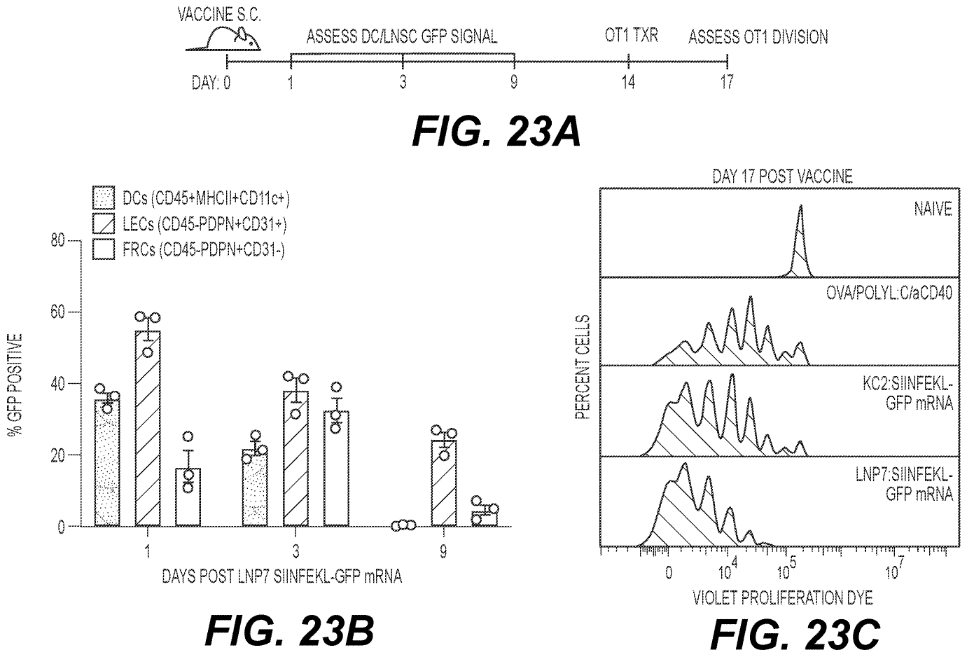

LNP encapsulated mRNA encoding SIINFEKL-GFP (2 ug RNA per injection site) or ovalbumin, polyI:C, anti-CD40 were administered subcutaneously (S.C) in a mouse footpad. An overview of the experimental design is shown as FIG. 23A. At indicated time-points after vaccination (1, 3, 9, 14, and 17 days) with LNP7 as the lipid coat, draining LNs were removed and digested prior to staining cells for markers of lymph node stromal cells (LNSC) and dendritic cells (DC)/monocytes as illustrated in FIG. 23B. GFP was only detected in DC populations (both migratory and LN resident) and LNSC populations indicated. At day 17 post vaccination with indicated LNPs that encapsulated the SIINFEKL-GFP mRNA, mice were euthanized and LNs removed and processed for detection of transferred violet proliferation dye VPD labeled OT1 CD8 T cells. Dilution peaks are shown in FIG. 23C of the VPD label with ova/polyI:C/aCD40 as a positive control of a vaccine that induces antigen archiving.

Protein antigen was detectable within dendritic cells (DC), LECs and FRCs early after vaccination with LNP7, with increased localization to LECs at day 1 (FIGS. 23A-23B). By 9 days post vaccination, the antigen was dominant in the LEC populations (FIG. 23B). Like protein antigens, protein antigens translated from mRNA-based vaccines were also detectable at late time points after vaccination as evaluated by the ability of CD8⁺ OT1 (SIINFEKL specific) T cells to respond to antigen 17 days post vaccination as assessed by the dilution of the dye label (FIG. 23C). Dilution of the dye label indicates that the mRNA antigen (SIINFEKL-GFP) specific CD8 T cells divided. Without wishing to be bound by theory, the more dilution of the dye generally indicates increased proliferation which is an indirect measurement of the amount of antigen the CD8 T cells respond to.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

Unless defined otherwise, all technical and scientific terms used herein have the same meanings as commonly understood by one of skill in the art to which the disclosed invention belongs. The following patents, applications and publications as listed below and throughout this document are hereby incorporated by reference in their entirety herein.
[1] WO2021/222903A2
[2] EP1922094B1
[3] CN105343015A
[4] US2008/0241256A1
[5] US2006/0034925A1
[6]U.S. Ser. No. 10/669,311B2
[7] TW201642850A
[8] US2017/0079916A1
[9] Tamburini, B. A., Burchill, M. A. and Kedl, R. M., 2014. Antigen capture and archiving by lymphatic endothelial cells following vaccination or viral infection. Nature communications, 5(1), p. 3989.
[10] Kedl, R. M., Lindsay, R. S., Finlon, J. M., Lucas, E. D., Friedman, R. S. and Tamburini, B. A. J., 2017. Migratory dendritic cells acquire and present lymphatic endothelial cell-archived antigens during lymph node contraction. Nature communications, 8(1), p. 2034. [11] Walsh, S. M., Sheridan, R. M., Lucas, E. D., Doan, T. A., Ware, B. C., Schafer, J., Fu, R., Burchill, M. A., Hesselberth, J. R. and Tamburini, B. A. J., 2021. Molecular tracking devices quantify antigen distribution and archiving in the murine lymph node. Elife, 10, p.e62781.

1. A lymphatic endothelial cell-specific lipid nanoparticle comprising a sterol, an ionizable lipid, a PEGylated lipid, and a phospholipid.

2. The lymphatic endothelial cell-specific lipid nanoparticle of claim 1, further comprising an alcohol.

3. (canceled)

4. (canceled)

5. The lymphatic endothelial cell-specific lipid nanoparticle of claim 1, wherein the phospholipid is 2-dioleoyl-sn-glycero-3-phosphoethanolamine (DOPE): 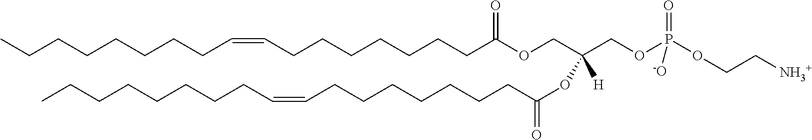
or
1,2-distearoyl-sn-glycero-3-phosphocholine (DSPC):

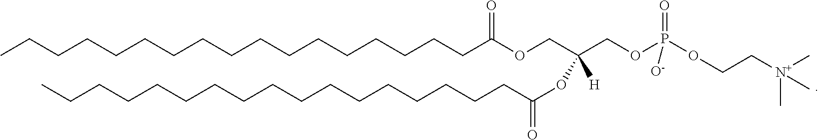 [SMILES: CCCCCCCCCCCCCCCCCC(=O)OC[C@H](COP(=O)([O-])OCC[N+](C)(C)C)OC(=O)CCCCCCCCCCCCCCCCC]

6. (canceled)

7. The lymphatic endothelial cell-specific lipid nanoparticle of claim 1, wherein the sterol comprises cholesterol.

8. (canceled)

9. The lymphatic endothelial cell-specific lipid nanoparticle of claim 1, wherein the ionizable lipid comprises 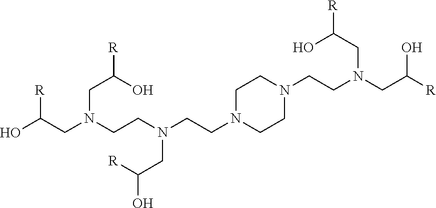
wherein each R is independently a substituted or unsubstituted C₈-C₁₈ alkyl.

10. (canceled)

11. (canceled)

12. The lymphatic endothelial cell-specific lipid nanoparticle of claim 1, wherein each R is independently an unsubstituted C₈-C₁₈ alkyl.

13. (canceled)

14. The lymphatic endothelial cell-specific lipid nanoparticle of claim 1, wherein the ionizable lipid is cKK-E12:

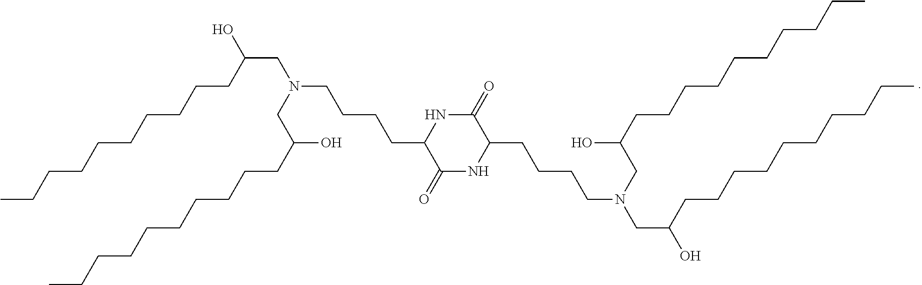 [SMILES: CCCCCCCCCCC(O)CN(CCCCC1NC(=O)C(CCCCN(CC(O)CCCCCCCCCC)CC(O)CCCCCCCCCC)NC1=O)CC(O)CCCCCCCCCC]

15. The lymphatic endothelial cell-specific lipid nanoparticle of claim 1, wherein the PEGylated lipid is C14PEG2000:

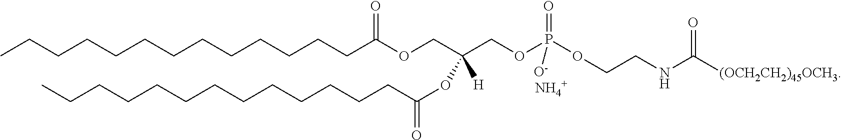

16. The lymphatic endothelial cell-specific lipid nanoparticle of claim 1, wherein a molar ratio of the ionizable lipid to the other components is from 25% to 60%.

17. (canceled)

18. The lymphatic endothelial cell-specific lipid nanoparticle of claim 1, wherein a molar ratio of the phospholipid to the other components is from 0.1% to 8%.

19. The lymphatic endothelial cell-specific lipid nanoparticle of claim 1, wherein a molar ratio of the PEGylated lipid to the other components is from 8% to 45%.

20. The lymphatic endothelial cell-specific lipid nanoparticle of claim 2, comprising from 30% to 50% v/v alcohol, from 12% to 25% v/v of the ionizable lipid, from 5% to 18% v/v of the sterol, from 5% to 8% v/v of the PEGylated lipid, and from 5% to 35% v/v of the phospholipid.

21. (canceled)

22. (canceled)

23. The lymphatic endothelial cell-specific lipid nanoparticle of claim 2, comprising:
44.88% v/v ethanol, 24.88% v/v cKK-E12, 13.56% v/v cholesterol, 6.76% v/v C14PEG2000, and 9.92% v/v DSPC, or
37.04% v/v ethanol, 17.04% v/v cKK-E12, 6.84% v/v cholesterol, 6.6% v/v C14PEG2000, and 32.48% v/v DOPE, or
43.96% v/v ethanol, 23.96% v/v cKK-E12, 5.35% v/v cholesterol, 6.18% v/v C14PEG2000, and 20.55% v/v DOPE.

24. (canceled)

25. (canceled)

26. (canceled)

27. (canceled)

28. A composition comprising: the lymphatic endothelial cell-specific lipid nanoparticle of claim 1 and a therapeutic agent, wherein the therapeutic agent comprises a nucleic acid, a peptide, or a small molecule.

29. (canceled)

30. (canceled)

31. The composition of claim 28, wherein the nucleic acid, peptide, or small molecule comprises, a VEGF protein, a nucleotide encoding a VEGF protein, or a fragment thereof.

32. (canceled)

33. (canceled)

34. (canceled)

35. (canceled)

36. (canceled)

37. (canceled)

38. (canceled)

39. (canceled)

40. A method of treating a lymphatics-associated disorder in a subject, comprising administering to the subject a therapeutically effective amount of the composition of claim 28.

41. (canceled)

42. (canceled)

43. A method of enhancing lymphangiogenesis in a subject, comprising administering to the subject a therapeutically effective amount of the composition of claim 28.

44. (canceled)

45. (canceled)

46. (canceled)

47. An mRNA vaccine comprising the lymphatic endothelial cell-specific lipid nanoparticle of claim 1 and an mRNA disposed therewithin.

48. (canceled)

49. A method of preventing or treating a disorder or an infection in a subject, comprising administering to the subject a therapeutically effective amount of the mRNA vaccine of claim 47.

50. (canceled)

51. (canceled)

52. A pharmaceutical composition comprising the composition of claim 28 and a pharmaceutically acceptable carrier.
